(19) United States
(12) Patent Application Publication
(10) Pub. No.: US 2024/0181173 A1
(43) Pub. Date: Jun. 6, 2024

(54) NEEDLE SAFETY SHEATH, GUARD, AND DEVICE

(71) Applicant: DENTAL PRODUCTS LAB, LLC, Seattle, WA (US)

(72) Inventors: Shan Eugene GAW, Seattle, WA (US); Robert Alexander RANDOLPH, Solana Beach, CA (US)

(73) Assignee: DENTAL PRODUCTS LAB, LLC, Seattle, WA (US)

(21) Appl. No.: 18/556,643

(22) PCT Filed: Apr. 21, 2022
(86) PCT No.: PCT/US2022/025804

§ 371 Date: Oct. 20, 2023

Related U.S. Application Data
(60) Provisional application No. 63/179,512, filed on Apr. 25, 2021.

(51) Int. Cl. A61M 5/32; A61M 5/34

(52) U.S. Cl. CPC A61M 5/3271; A61M 5/343; A61M 2005/3247; A61M 2210/0637

(57) ABSTRACT

The present disclosure is directed to embodiments of a needle safety sheath, guard, or device that is compatible and removably mountable to syringes (e.g., aspirating syringes for use within the dental industry) to which needles may be mounted. In at least one embodiment, the needle safety sheath, guard, or device includes one or more slots and one or more cutouts along a cylindrical safety sheath of the needle safety sheath, guard, or device along which the cylindrical safety sheath may slide from a retracted position (e.g., non-deployed state) to an extended position (e.g., deployed state) and vice versa. One or more flexible members may be along the cylindrical safety sheath that allow a medical practitioner to remove the needle from the syringe without the medical practitioner having to be exposed to a tip of the commercially available needle.

### BACKGROUND

### Technical Field

The present disclosure is directed to a needle safety sheath, guard, and device that is to be mounted to a syringe with a needle to administer a medication to a patient.

### Description of the Related Art

Generally, there is a broad range of needle safety devices on the market today that generally fit into two main categories. The first category includes products that remain detached from the syringe and needle requiring the user to bring the syringe and needle to the safety device or vice versa. The second category are those that are integrated into the syringe or needle, offering immediate protection at the point of use.

In utilizing the broad range of needle safety devices on the market today, there generally still is a common injury that occurs while a medical practitioner (e.g., dental practitioner, family-clinic practitioner, emergency room practitioner, etc.) is utilizing a syringe (e.g., an aspirating syringe by a dental practitioner) known as a needlestick or sharps injury in which the medical practitioner or user of the syringe accidentally stabs or jabs themselves with the needle on the syringe. For example, this needlestick or sharps injury may occur when recapping the needle of the syringe in between injections and/or prior to disposal as recapping the needle requires dexterity and close attention by the medical practitioner.

To avoid these needlestick or sharps injuries, an on-board needle safety device may provide an effective solution. Certain on-board needle safety devices that have been developed are integrated into the disposable needle or a major component of the syringe in which the syringe is functional only when joined together. In other words, these on-board needle safety devices are not compatible with commercially available syringes and needles. This results in medical practitioners or users of these needle safety devices to abandon their current syringes or needles and adopt new, unfamiliar equipment that is more expensive on a per patient basis than their current practice. This generally increases waste, costs, and expenses when replacing old equipment with new, unfamiliar equipment.

### BRIEF SUMMARY

The present disclosure is directed to embodiments of a needle safety sheath, guard, or device that reduces the likelihood of needlestick or sharps injuries. The embodiments of the needle safety sheath, guard, or device of the present disclosure include a retracted position in which a needle is exposed and an extended position in which the needle is fully covered. In other words, the embodiments of the needle safety sheath, guard, or device may be moved or actuated between the retracted position and the extended position, respectively.

In at least one embodiment of the present disclosure, a needle safety sheath, guard, or device is structured and configured to be mounted to a syringe on which a needle assembly may be mounted such that a medical practitioner (e.g., dentist, doctor, nurse, etc.) may surround the needle with the needle safety sheath, guard, or device when the syringe and a needle of the needle assembly are not in use. The needle safety sheath, guard, or device may be mounted to the syringe in a separable manner such that the needle safety sheath, guard, or device may be removed and separated from the syringe. The syringe may be a commercially available aspirating syringe and secured under the hub of a standard, commercially available needle. The needle is threaded onto the body of an aspirating syringe. During a procedure requiring more than one injection, the medical practitioner can slide the embodiment of the needle safety sheath, guard, or device to the extended position to fully cover the needle in between injections. At the time of disposal of the needle, the embodiment of the needle safety sheath, guard, or device is extended in an extended position in the same manner as when the needle safety sheath, guard, or device is fully covering the needle. At the time of disposal of the needle, one or more flexible members of the embodiment of the needle safety sheath, guard, or device allow the medical practitioner to engage the hub of the needle at which point the medical practitioner unthreads the hub of the needle from the syringe separating the needle from the syringe. Once the needle is separated from the syringe, the medical practitioner continues to apply pressure to the flexible members such that the needle remains secured inside of the embodiment of the needle safety sheath, guard, or device, allowing the medical practitioner to then dispose of the needle into a needle or sharps disposal container by releasing the flexible members to deposit the needle assembly into the needle or sharps disposal container. Once the needle is disposed, the safety sheath may then be prepared for sterilization and reused using the same standard materials and processing equipment used for syringes and other reusable tools common to general medical practices and common to dentistry practices.

### BRIEF DESCRIPTION OF THE SEVERAL VIEWS OF THE DRAWINGS

For a better understanding of the embodiments, reference will now be made by way of example to the accompanying drawings. In the drawings, identical reference numbers identify the same or similar elements or acts unless the context indicates otherwise. The sizes and relative proportions of the elements in the drawings are not necessarily drawn to scale. For example, some of these elements may be enlarged and positioned to improve drawing legibility.

FIG. 1 is a perspective, exploded view of a system including a syringe, a needle, a carpule, and an embodiment of a needle safety sheath, guard, or device system of the present disclosure;

FIG. 2A is a perspective view of the system as shown in FIG. 1 with the embodiment of the needle safety sheath, guard, or device of the present disclosure in a retracted position (e.g., a non-deployed state);

FIG. 2B is a perspective view of the system as shown in FIG. 1 with the embodiment of the needle safety sheath, guard, or device of the present disclosure in an extended position (e.g., a deployed state);

FIG. 2C is a perspective view of the system as shown in FIG. 1 with the embodiment of the needle safety sheath, guard, or device of the present disclosure in a locked position when in the extended position (e.g., the deployed state);

FIG. 3A is a side view of the embodiment of the needle safety sheath, guard, or device of the present disclosure removably mounted to the syringe;

FIG. 3B is a side view of the embodiment of the needle safety sheath, guard, or device of the present disclosure removably mounted to the syringe;

FIG. 4A is a side view of the embodiment of the needle safety sheath, guard, or device of the present disclosure removably mounted to the syringe and the needle removably coupled to the syringe;

FIG. 4B is a side view of the embodiment of the needle safety sheath, guard, or device of the present disclosure removably mounted to the syringe and the needle removably coupled to the syringe;

FIG. 5A is a perspective view of the embodiment of the needle safety sheath, guard or device of the present disclosure;

FIG. 5B is a right side view of the embodiment of the needle safety sheath, guard, or device of the present disclosure;

FIG. 5C is a front side view of the embodiment of the needle safety sheath, guard, or device of the present disclosure;

FIG. 5D is a left side view of the embodiment of the needle safety sheath, guard, or device of present disclosure;

FIG. 5E is a rear side view of the embodiment of the needle safety sheath, guard, or device of the present disclosure;

FIG. 5F is a bottom side view of the embodiment of the needle safety sheath, guard, or device of the present disclosure;

FIG. 5G is a top side view of the embodiment of the needle safety sheath, guard or device of the present disclosure;

FIG. 6 is a perspective exploded view of the needle safety sheath, guard, or device;

FIG. 7A is a perspective view of an internal base member of the needle safety sheath, guard, or device;

FIG. 7B is a top plan view of the internal base member as shown in FIG. 7A;

FIG. 7C is a bottom plan view of the internal base member as shown in FIG. 7A;

FIG. 8A is a perspective view of a first portion of a cylindrical body of the needle safety sheath, guard, or device;

FIG. 8B is a top plan view of the first portion of the cylindrical body as shown in FIG. 8A;

FIG. 8C is a bottom plan view of the first portion of the cylindrical body as shown in FIG. 8A;

FIG. 9A is a perspective view of a second portion of the cylindrical body of the needle safety sheath, guard, or device;

FIG. 9B is a top plan view of the second portion of the cylindrical body as shown in FIG. 9A;

FIG. 9C is a bottom plan view of the second portion of the cylindrical body as shown in FIG. 9A;

FIG. 10 is directed to a flowchart of an embodiment of a method of the present disclosure to install the embodiment of the needle sheath, guard, or device of the present disclosure on the syringe, to install a needle assembly on the syringe, and to install a carpule containing a medication within the syringe;

FIGS. 11A-11G are views of performing the embodiment of the method of the flowchart as shown in FIG. 10;

FIG. 12 is directed to a flowchart of an embodiment of a method of the present disclosure to dispose of the needle assembly on the syringe by utilizing the embodiment of the needle safety sheath, guard, or device of the present disclosure on the syringe to remove the needle assembly; and

FIG. 13A-13F are views of performing the embodiment of the method of the present disclosure of the flowchart as shown in FIG. 12.

### DETAILED DESCRIPTION

In the following description, certain specific details are set forth in order to provide a thorough understanding of various embodiments of the disclosure. However, one skilled in the art will understand that the disclosure may be practiced without these specific details. In other instances, well-known structures associated with syringes, medical devices, and dentistry devices have not been described in detail to avoid unnecessarily obscuring the descriptions of the embodiments of the present disclosure.

Unless the context requires otherwise, throughout the specification and claims that follow, the word “comprise” and variations thereof, such as “comprises” and “comprising,” are to be construed in an open, inclusive sense, that is, as “including, but not limited to.”

The use of ordinals such as first, second, third, etc., does not necessarily imply a ranked sense of order, but rather may only distinguish between multiple instances of an act or a similar structure or material.

Reference throughout this specification to “one embodiment” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The terms “top,” “bottom,” “upper,” “lower,” “left,” “right,” “rear,”or other like locating words are used for only discussion purposes based on the orientation of the components in the discussion of the figures in the present disclosure as follows. These terms are not limiting as to the possible positions explicitly disclosed, implicitly disclosed, or inherently disclosed in the present disclosure.

The terms “vertical,” “horizontal,” or other like orientation words are used for only discussion purposes based on the orientation of the components in the discussion of the figures in the present disclosure as follows. These terms are not limiting as to the possible positions and orientations explicitly disclosed, implicitly disclosed, or inherently disclosed in the present disclosure.

The term “substantially” is used to clarify that there may be slight differences and variations when a package is manufactured in the real world, as nothing can be made perfectly equal or perfectly the same. In other words, “substantially” means and represents that there may be some slight variation in actual practice and instead is made or manufactured within selected tolerances.

As used in this specification and the appended claims, the singular forms “a,” “an,” and “the” include plural referents unless the content clearly dictates otherwise.

While at least one embodiment of a needle safety sheath, guard, or device is shown and described within the present disclosure, it will be readily appreciated that embodiments of the needle safety sheath, guard, or device are not limited thereto. In various embodiments, the structures, devices, methods and the like described herein may be embodied in or otherwise utilized in any suitable type or form of a needle safety sheath, guard, or device, and may be manufactured utilizing any suitable manufacturing technologies to manufacture embodiments of the needle safety sheath, guard, or device of the present disclosure.

The present disclosure is directed to embodiments of a needle safety sheath, guard, or device that reduces the likelihood of needlestick or sharps injuries. The embodiments of the needle safety sheath, guard, or device of the present disclosure include a retracted position in which a needle is exposed and an extended position in which the needle is fully covered. The embodiments of the needle safety sheath, guard, or device may be moved or actuated between the retracted positions and the extended positions, respectively. The embodiments of the needle safety sheath, guard, or device may be locked into positon when in the retracted positions and the extended positions, respectively.

In at least one embodiment of the present disclosure, a needle safety sheath, guard, or device is structured and configured to be mounted to a syringe. In the at least one embodiment, the safety sheath, guard, or device is separable from the syringe. For example, the needle safety sheath, guard, or device may initially be mounted to the syringe and then removed from the syringe such that the needle safety sheath, guard, or device is removed without damaging the syringe. In the at least one embodiment of the present disclosure, when a needle, which may be part of a needle assembly, and the needle safety sheath, guard, or device are on the syringe, a medical practitioner (e.g., dentist, doctor, nurse, etc.) may surround the needle with the needle safety sheath, guard, or device when the syringe and the needle are not in use. The syringe may be a commercially available aspirating syringe and secured under a hub of a standard, commercially available needle. The needle is threaded onto a syringe body of an aspirating syringe. During a procedure requiring more than one injection, the medical practitioner can slide the embodiment of the needle safety sheath, guard, or device to the extended position to fully cover the needle in between injections. At the time of disposal of the needle, the embodiment of the needle safety sheath, guard, or device is extended in an extended position in the same manner as when the needle safety sheath, guard, or device is fully covering the needle. At the time of disposal of the needle, one or more flexible members of the embodiment of the needle safety sheath, guard, or device allow the medical practitioner to engage the hub of the needle at which point the medical practitioner unthreads the hub of the needle from the syringe separating the needle from the syringe. Once the needle is separated from the syringe, the medical practitioner continues to apply pressure to the flexible members such that the needle remains secured inside of the embodiment of the needle safety sheath, guard, or device, allowing the medical practitioner to then release the flexible members to deposit the needle into the needle or sharps disposal container. The safety sheath can then be prepared for sterilization and reuse using the same standard materials and processing equipment used for syringes and other reusable instruments common to general medical practices and common to dentistry practices.

These various embodiments of the needle safety sheath, guard, or device of the present disclosure may all be utilized with commercially available syringes and commercially available needles with needle hubs. In other words, these various embodiments of the needle safety sheath, guard, or device may be retrofitted onto the already commercially available syringes and commercially available needles with needle hubs to avoid medical practitioners (e.g., dentists, doctors, nurses, etc.) from having to purchase or obtain new syringes and needles. As the needle safety sheath, guard, or device may be utilized with the commercially available syringe and needles with needle hubs, the medical practitioners may utilize medical equipment that the medical practitioners or the users are already familiar with such that the medical practitioners do not have to become familiar with new medical equipment. As the needle safety sheath, guard, or device may be utilized with the commercially available syringe and needles with needle hubs, the medical practitioners or users do not have to purchase any new equipment such that costs remain low relative to other conventional needle safety sheaths, guards, or devices that are integrated within conventional syringes or conventional needles.

Alternatively, the various embodiments of the needle safety sheath, guard, or device of the present disclosure may be utilized with custom syringes, custom needles, or both that may be customized based on medical practitioner preferences or specialized uses. In other words, the various embodiments of the needle safety sheath, guard or device of the present disclosure may be adapted and restructured to be utilized with various types of syringes, various types of needles, or both, which may have different shapes and sizes.

These conventional needle safety sheaths, guards, or devices integrated with the conventional syringe and conventional needles are not removable from either the conventional syringe or conventional needles such that the conventional needle safety sheaths, guards, or devices are disposed of along with either the conventional syringe, the conventional needle, or both. This increases waste costs as the conventional needle safety sheaths, guards, or devices may not be re-utilized by being re-sterilized but are instead simply thrown out and become waste. This additional waste results in additional expenses and costs and creates more waste that is generally bad for the environment. These conventional needle safety sheaths, guards, or devices result in the medical practitioner having to utilize a foreign syringe or needle during injections that the medical practitioner is not familiar with, which may increase the likelihood of a needlestick injury occurring as a result of the medical practitioner being inexperienced with utilizing the foreign syringe or needle.

Unlike the conventional needle safety sheaths, guards, or devices that become waste as the conventional needle safety sheaths, guards, or devices cannot be re-utilized and re-sterilized, the various embodiments of the needle safety sheath, guard, or devices of the present disclosure are separate and distinct from syringes to which the various embodiments of the needle safety sheaths, guards, or devise may be mounted. The various embodiments of the needle safety sheaths, guards, or devices of the present disclosure are removably mounted to the syringes and include one or more elastically deformable and flexible members that may be utilized in removing a needle hub in which a needle is present from the syringe. Embodiments of the needle safety sheaths, guards, or devices of the present disclosure may avoid the issues of the conventional needle safety sheaths, guards, or devices as discussed above. For example, the embodiments of the needle safety sheaths, guards, or devices may be retrofitted or mounted onto various types of aspirating syringes that allow for the medical practitioner simply to mount one of the embodiments of the needle safety sheaths, guards, or devices of the present disclosure to a type of aspirating syringe that the medical practitioner is familiar with and experienced in utilizing, which may reduce the likelihood of a needlestick injury occurring. Embodiments of the needle safety sheaths, guards, or devices of the present disclosure are removable from the various types of aspirating syringes to which the embodiments of the needle safety sheaths, guards, or devices of the present disclosure may be mounted, which may allow the embodiments of the needle safety sheaths, guards, or devices of the present disclosure to be removable and re-sterilized such that the embodiments of the needle safety sheaths, guards, or devices may be reutilized and reused. This removability, re-utilization, and re-sterilization of the embodiments of the needle safety sheaths, guards, or devices of the present disclosure may reduce waste, and, therefore, reduce waste costs and expenses incurred by the medical practitioner.

The various embodiments of the needle safety sheaths, guards, or devices being removably mounted from the syringes allows for the various embodiments of the needle safety sheaths, guards, or devices of the present disclosure to be removed such that they can be re-sterilized after use and re-utilized. The various embodiments may be structured and configured to be removed from the syringes without damaging the syringes. The one or more elastically deformable and flexible members of the various embodiments of the needle safety sheaths, guards, or devices allows for a medical practitioner (e.g., dentist, doctor, nurse, etc.) or a user to remove the needle hubs in which the needles are present from the syringe without having to be exposed to tips of the needles. As the medical practitioner or the user is not exposed to the tips of the needles when disposing of the needles, the likelihood of needlestick or sharp injuries is reduced.

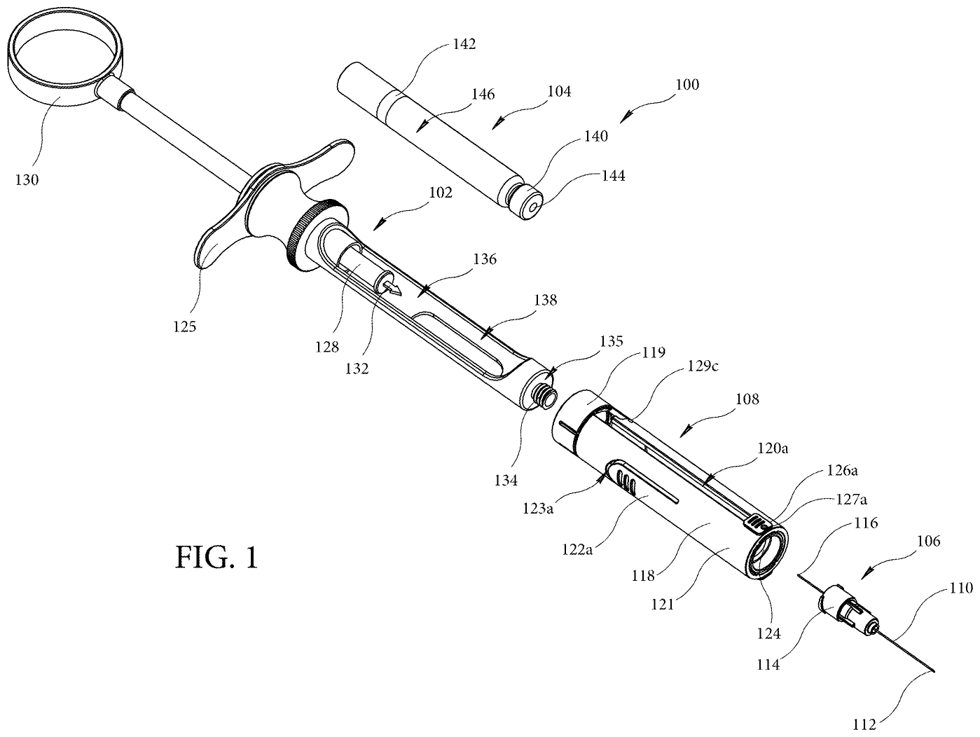

FIG. 1 is an exploded isometric view illustrating an injection system 100, which may be a dental injection system. The injection system 100 includes a syringe 102, a carpule 104, a needle assembly 106, and a safety device 108, which may also be referred to as a safety structure. The safety device 108 may be referred to as a safety guard, a safety cover, a safety sheath, or some other similar or like reference to the safety device 108. The safety device 108 may be configured to be removably connected or mounted to the syringe 102 such that the safety device 108 is separable and removable from the syringe 102 to allow the safety device 108 to be reused or replaced before, during, or after a procedure on a patient. For example, the safety device 108 may be removed from the syringe 102 for re-sterilization of the safety device 108 for re-utilization on a new or unused syringe for a new procedure on another patient after a previous procedure was completed on a previous patient.

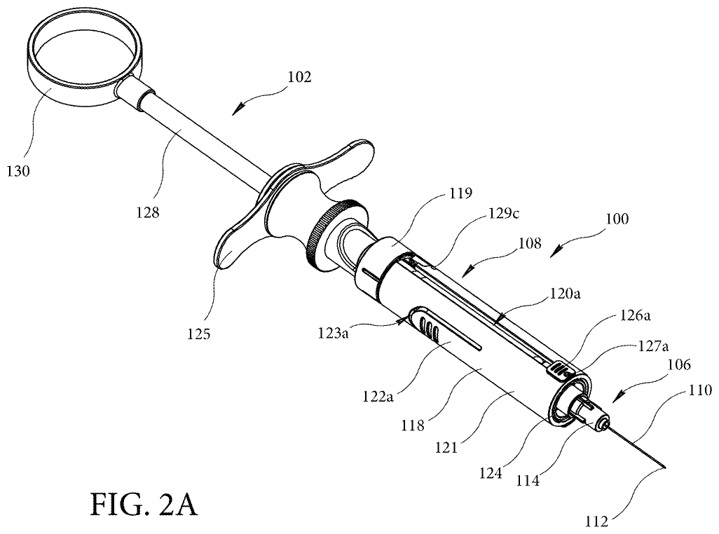

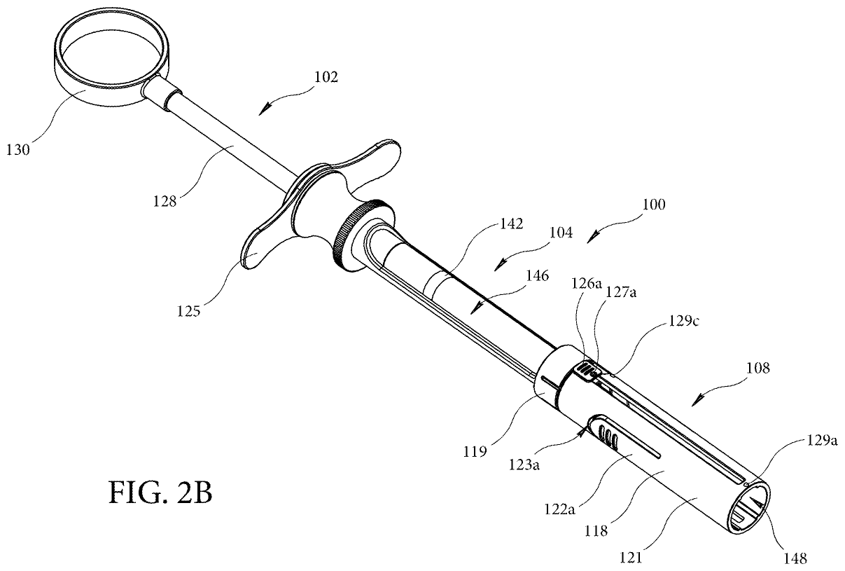

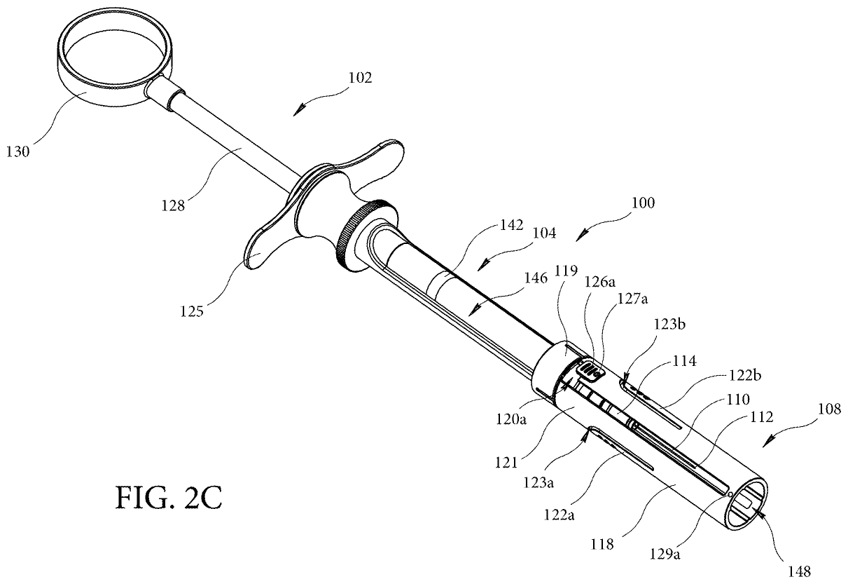

The safety device 108 is configured to overlap or be adjacent to the syringe 102 when the needle 110 is exposed to conduct an injection procedure (e.g., injecting a medication into a patient present within the carpule 104 utilizing the dental injection system 100). For example, the safety device 108 is shown in a retracted position as shown in FIG. 2A of the present disclosure. When the safety device 108 is in the retracted position, a needle 110 of the needle assembly 106 is readily accessible and an injection tip 112 of the needle 110 is exposed from the safety device 108.

A needle hub 114 of the needle assembly 106 holds the needle 110 of the needle assembly 106. The needle 110 extends through the needle hub 114 such that a carpule tip 116 of the needle 110 is on an opposite side of the needle hub 114 relative to the injection tip 112. In other words, the needle hub 114 is present along the needle 110 and is between the injection tip 112 and the carpule tip 116. The injection tip 112 is configured to be inserted into a patient to inject medication in the carpule 104 into the patient, and the carpule tip 116 is configured to be inserted into and through a diaphragm 144 of a stopper 140 such that the medication within the carpule 104 may be in fluid communication with the injection tip 112 of the needle 110 through the carpule tip 116. In some instances such as within the medical industry, the needle hub 114 and the needle 110 of the needle assembly 106 may be referred to together as a “needle.”

The safety device 108 may be configured to extend around the needle 110 of the needle assembly 106 in between multiple and successive injection procedures and during a needle disposal process to reduce the likelihood of needlestick injuries. For example, the safety device 108 is shown in an extended position as shown in FIGS. 2B and 2C. When the safety device 108 is in the extended position, the needle 110 is not readily accessible and access to the injection tip 112 of needle 110 is prevented, stopped, or blocked by the safety device 108.

In some embodiments, the safety device 108 includes a cylindrical body 118 with two opposing slots 120a, 120b that extend along the cylindrical body 118, two opposing flexible and elastically deformable grip elements 122a, 122b, which may be referred to as two opposing flexible and elastically deformable members, that are present along the cylindrical body 118, an internal base member 124 that is within the cylindrical body 118, and two interfacing tab elements 126a, 126b coupled to or integral to the internal base member are present along one of the opposing slots 120a, 120b. For example, the tab elements 126a, 126b may be part of a single, continuous material of the internal base member 124. The tab elements 126a, 126b may be referred to as tabs, tab structures, or some other similar or like reference to the tab elements 126a, 126b. The cylindrical body 118 may be referred to as a sheath, a guard, a guard sheath, or some other similar or like reference to the cylindrical body 118.

The cylindrical body 118 may include a first portion 119 and a second portion 121 that are coupled together. In some embodiments, the first portion 119 and the second portion 121 may be threadedly engaged with each other to couple the first portion 119 to the second portion 121. In some embodiments, the first portion 119 and the second portion may be snap fit together to couple the first portion 119 to the second portion 121. The first portion 119 may be referred to as a ring portion, a first cylindrical portion, or some other similar or like reference to the first portion 119. The second portion 121 may be referred to as a sheath, a guard, a sheath portion, a guard portion, a barrier portion, or some other similar or like reference to the second portion 121.

In an alternative embodiment, the first portion 119 and the second portion 121 may instead be a single piece of material such that the first and second portions 119, 121 are integral to each other and made of a single, continuous piece of material. For example, this alternative embodiment in which the first and second portions 119, 121 are made of a single, continuous material may be formed by an injection molding process or some other type of manufacturing process in which the first and second portions 119, 121 may be formed as a single, continuous piece of material.

In some embodiments, when the first and second portions 119, 121 of the cylindrical body 118 are formed as a single, continuous piece of material, the internal base member 124 may be made, formed, fabricated, assembled, manufactured, or inserted to be present within the single, continuous piece of material of the cylindrical body 118 in a similar fashion as shown in FIGS. 2A-2C, 5F, 5G, and 6 of the present disclosure. For example, the first and second portions 119, 121 of the cylindrical body 118 and the internal base member 124 may be formed in a molding process such that the internal base member 124 is assembled within the cylindrical body 118 when the first and second portions 119, 121 are made of a single, continuous material and are integral to each other.

Two opposing slots 123a, 123b, respectively, are directly adjacent to one of the two opposing flexible and elastically deformable grip elements 122a, 122b, respectively. The two opposing slots 123a, 123b extend through the second portion 121 of the cylindrical body 118 such that the two opposing slots 123a, 123b are adjacent to a corresponding one of the two flexible and elastically deformable grip elements 122a, 122b, respectively.

In some alternative embodiments, the two opposing slots 123a, 123b may not be present and instead the two opposing flexible and elastically deformable grip elements 122a, 122b may be some other type of flexible and elastically deformable structure that is configured to be flexible and elastically deformable inward in a similar or like fashion as the two opposing flexible and elastically deformable grip elements 122a, 122b. For example, in at least one alternative embodiment, the first and second deformable grip elements 122a, 122b may be replaced with indentations or dimples that may be elastically deformable inward towards the needle assembly 106 such that the inner surface aligned with the indentations or dimples may abut and contact the needle assembly 106 such that the needle assembly 106 may be removed from the syringe 102 utilizing the indentations or dimples. For example, in another alternative embodiment, the two opposing slots 123a, 123b may not be present and the two opposing flexible and elastically deformable grip elements 122a, 122b may be replaced with rubber elements that are flexible and elastically deformable inward such that inner surfaces or structures of the rubber elements abut and contact the needle assembly 106 such that the needle assembly 106 may be removed from the syringe 102 utilizing the rubber elements. In some alternative embodiments, the rubber elements may be made of some other type of elastically deformable material.

In some alternative embodiments, the two opposing slots 123a, 123b and the two opposing flexible and elastically deformable grip elements 122a, 122b may be replaced. For example, in at least one alternative embodiment, the two opposing slots 123a, 123b and the two opposing flexible and elastically deformable grip elements 122a, 122b may be replaced by two opposing openings, and a tool with two opposite ends may be inserted into the two opposing openings. The two opposite ends of the tool may mechanically engage with the needle assembly 106 when the alternative embodiment of the safety device 108 is in the extended position as shown in FIG. 2C to remove the needle assembly 106 from the alternative embodiment of the safety device 108.

Alternatively, instead of the tool as described above, a sharps container may include a needle assembly removal structure to remove the needle assembly 106 from the syringe 102. For example, the needle assembly removal structure of the sharps container may include two opposite structures that may be inserted into two opposing openings of the needle safety sheath, guard, or device such that the two opposite structures mechanically engage with the needle assembly 106. The two opposite ends of the needle assembly removal structure of the sharps container may mechanically engage with the needle assembly 106 when the alternative embodiment of the safety device 108 is in the extended position as shown in FIG. 2C to remove the needle assembly from the alternative embodiment the needle safety sheath, guard, or device.

In view of the above discussion of these alternative embodiments, it will be readily appreciated that various types of features and structures may be utilized to remove the needle assembly 106 from the syringe 102 when the cylindrical body 118 of the safety device 108 is in the extended position as shown in shown in FIG. 2C.

In view of the above discussion, the deformable grip elements 122a, 122b, the dimples or indentations, the rubber elements, or other similar or like types of structures that may be part of, coupled to, on, or accessible at an exterior of the cylindrical body 118 to remove the needle assembly 106 may be referred to as interface or engagement elements or structures.

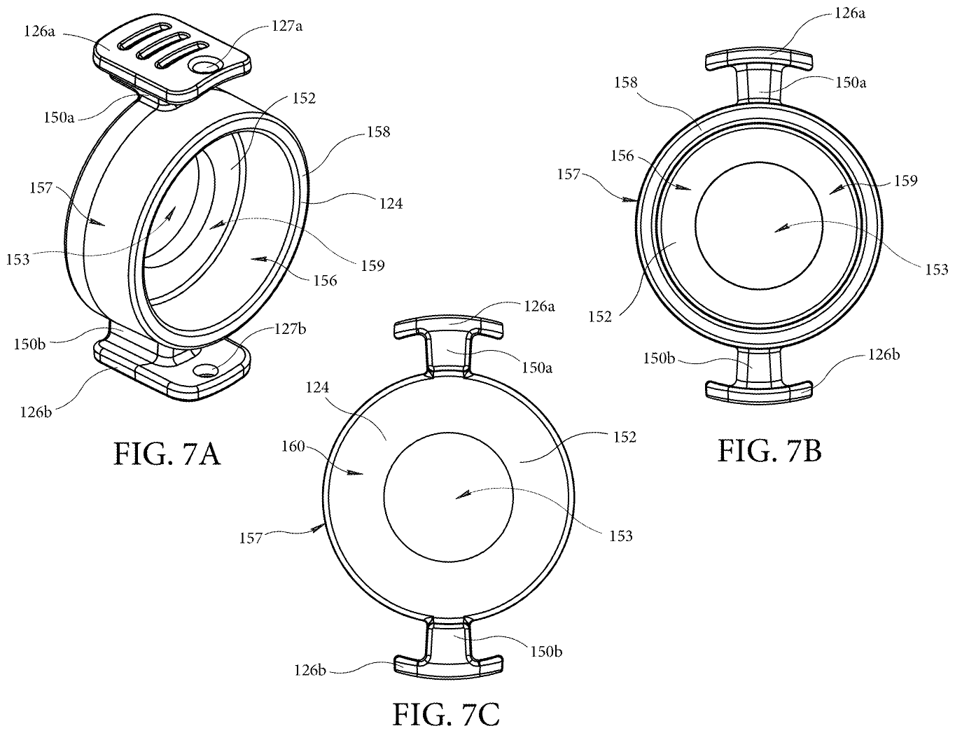

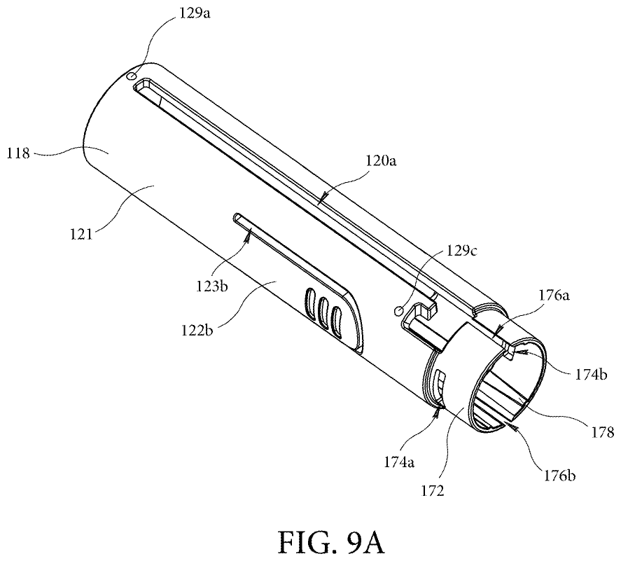

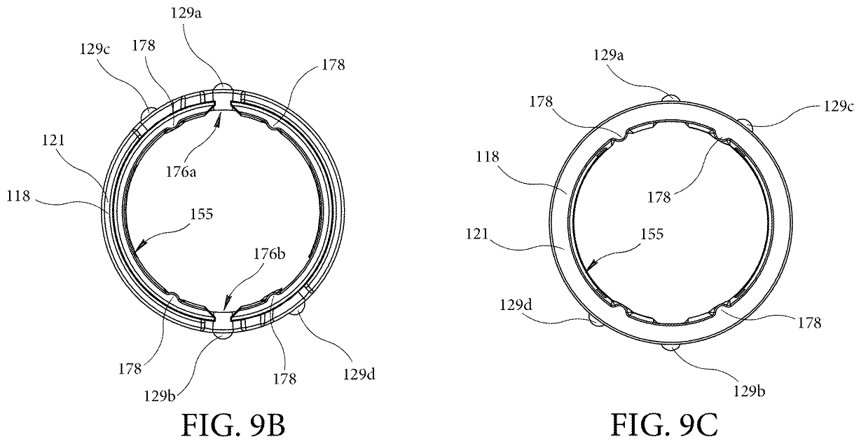

The tab elements 126a, 126b of the internal base member 124 remain fixed in position relative to the syringe 102 and the needle assembly 106 when the safety device 108 is mounted to the syringe 102. The pathway slots 120a, 120b allow the cylindrical body 118 of the safety device 108 to move or slide along a central axis of the syringe 102 to either expose the injection tip 112 of the needle 110 when in the retracted position (e.g., see retracted position as shown in FIG. 2A of the present disclosure) or extend around and cover the injection tip 112 of the needle 110 when in the extended position (e.g., see extended positions as shown in FIGS. 2B and 2C of the present disclosure). Each of the tab elements 126a, 126b includes a detent recess or opening 127a, 127b (see FIG. 7A of the present disclosure) in which detent balls 129a, 129b, 129c, 129d (see FIGS. 9B and 9C of the present disclosure) may be inserted. The detent balls 129a, 129b, 129c, 129d may be semi-spheres that are configured to readily slide into the detent openings 127a, 127b and slide out of the detent openings 127a, 127b when enough force is applied to overcome interlocking forces of the detent balls 129a, 129b, 129c, 129d and the detent openings 127a, 127b. When the cylindrical body 118 is in the retracted position as shown in FIG. 2A, the cylindrical body 118 is in an undeployed state in which the injection tip 112 of the needle 110 is exposed and readily accessible for injecting a patient. When the cylindrical body 118 is in the extended position as shown in FIGS. 2B and 2C, the cylindrical body 118 is in a deployed state in which the injection tip 112 of the needle 110 is covered and is not readily accessible, and, instead, the cylindrical body 118 prevents, blocks, or stops one accessing or being exposed to the injection tip 112 of the needle 110.

When in the extended position as shown in FIG. 2B, the flexible and elastically deformable grip elements 122a, 122b integrated in the cylindrical body 118 of the safety device 108 may contact the needle hub 114 of the needle assembly 106 by applying pressure to the grip elements 122a, 122b flexing and depressing the grip elements 122a, 122b inward towards the needle hub 114. Once enough pressure is applied such that the grip elements 122a, 122b of the safety device 108 contact the needle hub 114, the safety device 108 or the syringe 102 may be rotated while the grip elements 122a, 122b are in contact with the needle hub 114 such that the needle hub 114 of the needle assembly 106 rotates and is removed from a threaded structure 134 of the syringe 102. In other words, the needle hub 114 is threadedly disengaged with the threaded structure 134 of the syringe 102. Threadedly engaging the needle assembly 106 with the threaded structure 134 may include turning the needle assembly 106 or the syringe 102 by a quarter (¼) turn, a half (½) turn, or some other turn amount to threadedly engage the needle assembly 106 with the syringe 102. The needle assembly 106 may remain inside of the cylindrical body 118 of the safety sheath 108 until the needle assembly 106 is disposed into a sharps safety receptacle (e.g., a sharps container, a sharps bin, a sharps compartment, etc.) to reduce the likelihood of the medical practitioner being exposed to the injection tip 112 and the carpule tip 116 of the needle 110 of the needle assembly 106. In other words, the cylindrical body 118 may be positioned in the extended position to cover the injection tip 112 of the needle 110 when the injection system 100 is not being utilized to inject a patient, and the cylindrical body 118 covers the injection tip 112 and the carpule tip 116 of the needle 110 when the needle assembly 106 is being disposed of in a sharps safety receptacle utilizing the safety device 108.

While in the embodiment as shown in FIGS. 1, 2A, 2B, and 2C, the grip elements 122a, 122b are shown as being integral to the cylindrical body 118, in some alternative embodiments, the grip elements 122a, 122b may be separate and distinct components that are instead fastened or coupled to the cylindrical body 118. For example, in at least one alternative embodiment, when the grip elements 122a, 122b are separate and distinct components from the cylindrical body 118, the grip elements 122a, 122b may each include male snap fit fastener structures that are inserted into female snap fit fastener structures accessible at an exterior of the cylindrical body 118. These male snap fit fastener structures of the grip elements 122a, 122b may be inserted into the female snap fit fastener structures of the cylindrical body 118 such that the grip elements 122a, 122b are fastened or coupled to the cylindrical body 118.

While in the embodiment as shown in FIGS. 1, 2A, 2B, and 2C, the needle assembly 106 is coupled to the syringe 102 utilizing the threaded structure 134, in some alternative embodiments, the threaded structure 134 may be replaced with some other type of structure to fasten or couple the needle assembly 106 to the syringe 102. For example, in at least one alternative embodiment, the threaded structure 134 may be replaced with a female snap fit structure and the needle assembly 160 may include a male snap fit structure. The male snap fit structure of the needle assembly 106 may be inserted into the female snap fit structure of the syringe 102 such that the needle assembly 106 is fastened to the syringe 102. Alternatively, the needle assembly 106 may include a female snap fit structure and the syringe 102 may include a male snap fit structure instead of the threaded structure 134. In other words, alternative structures of fastening techniques may be utilized to fasten or couple the needle assembly 106 to the syringe 102.

The syringe 102 includes an optional finger bar structure 125 through which a piston rod 128 extends. A ring structure 130 is at a first end of the piston rod 128 and a harpoon structure 132 at a second end of the piston rod 128 opposite to the first end of the piston rod 128. The ring structure 130 is configured to, during operation, be utilized to move the piston rod 128 towards and away from the threaded structure 134 at an end of the syringe 102 that is opposite to an end at which the ring structure 130 is present. An opening 136 is accessible at an exterior of the syringe 102 and provides access to a cavity 138 within the syringe. The opening 136 may be referred to as a carpule opening and the cavity 138 may be referred to as a carpule cavity. While the syringe 102 as shown in the present disclosure shows the optional finger bar structure 125, in some alternative embodiments of the syringe 102, the syringe 102 may not include the optional finger bar structure 125 as the syringe 102 may be a different type of syringe or a different type of aspirating syringe than the embodiment as shown in the present disclosure. In other words, various different types of syringes or aspirating syringes with varying structures may be utilized with the various embodiments or alternative embodiments of the safety device 108 within the scope of the present disclosure.

The carpule 104 includes the stopper 140 at an end of the carpule 104 and a plunger 142, which may be rubber or silicone. The stopper 140 is at an end of the carpule 104 opposite to the end of the carpule 104 at which the stopper 140 is present. The stopper 140 includes the diaphragm 144, which may be rubber or silicone. The diaphragm 144 is centrally located along the stopper 140. A medication cavity 146 is present between the stopper 140 and the plunger 142. The medication cavity 146 is configured to store a medication that may be injected into a patient before, after, or during a procedure. For example, the medication stored within the medication cavity 146 may be a pain reducer, an anesthetic, or some other type of medication that may be injected into a patient.

When loading the syringe 102 with the carpule 104, the carpule 104 may be inserted into the cavity 138 by passing the carpule through the opening 136 into the cavity 138. Once the carpule 104 is present within the cavity 138, the piston rod 128 is moved towards the carpule 104 such that the harpoon structure 132 engages the plunger 142. For example, the plunger 142 may include an opening (not shown) that is structured to receive the harpoon structure 132 such that the harpoon structure 132 interlocks with the plunger 142. Once the harpoon structure 132 interlocks with the plunger 142, a medical practitioner or a user of the injection system 100 may push or pull on the ring 130 to move the piston rod 128 towards and away from the threaded structure 134 of the syringe 102 such that the plunger 142, which is interlocked with the harpoon structure 132, moves towards and away from the threaded structure 134 along with the piston rod 128. An exterior surface 135 is adjacent to the threaded structure 134 and extends around the threaded structure 134.

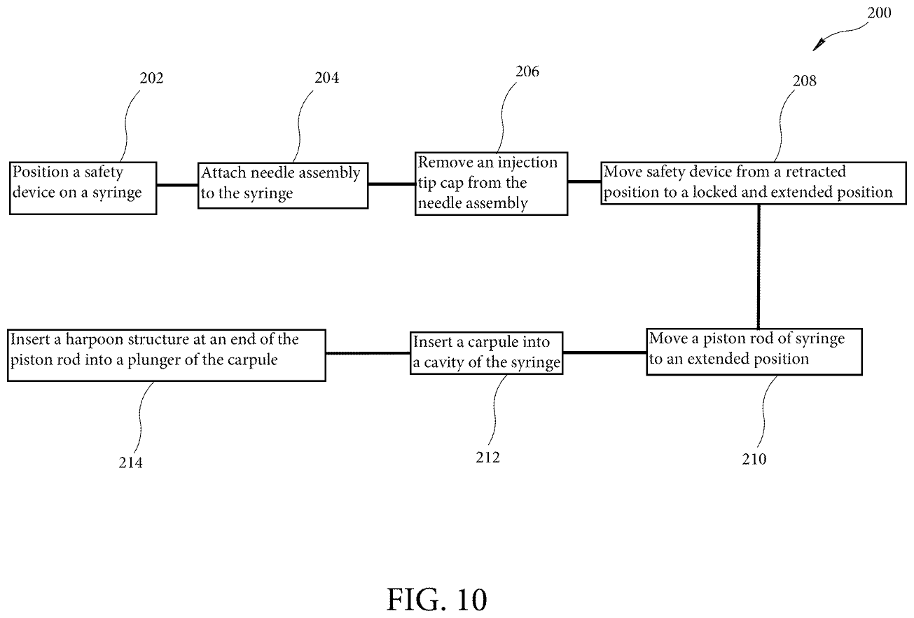

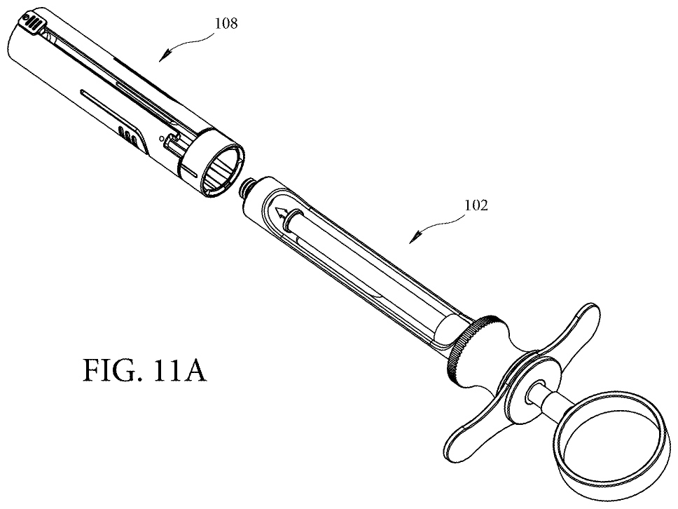

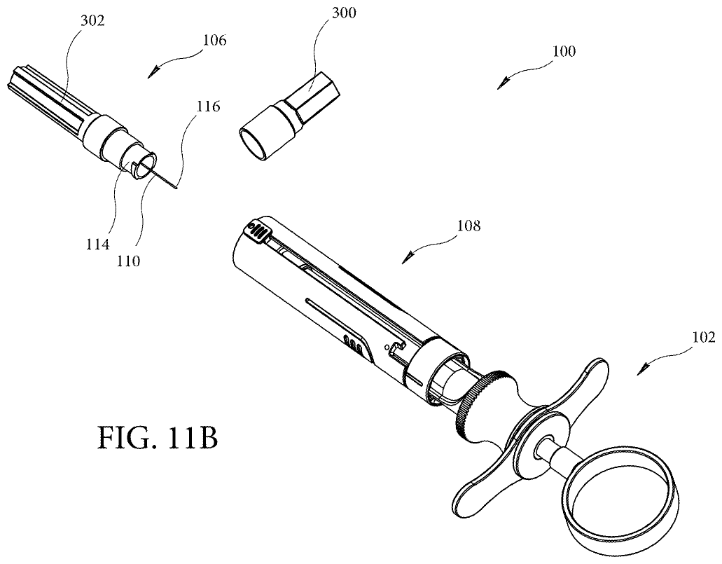

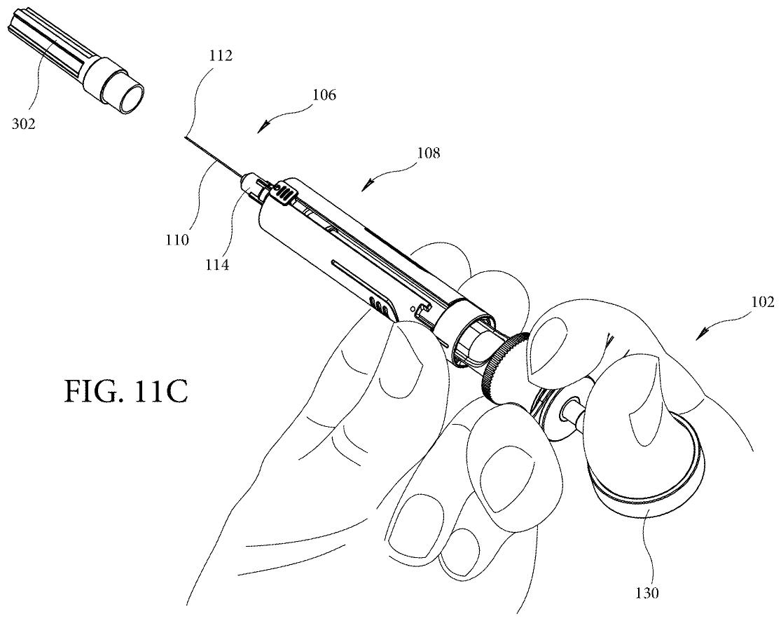

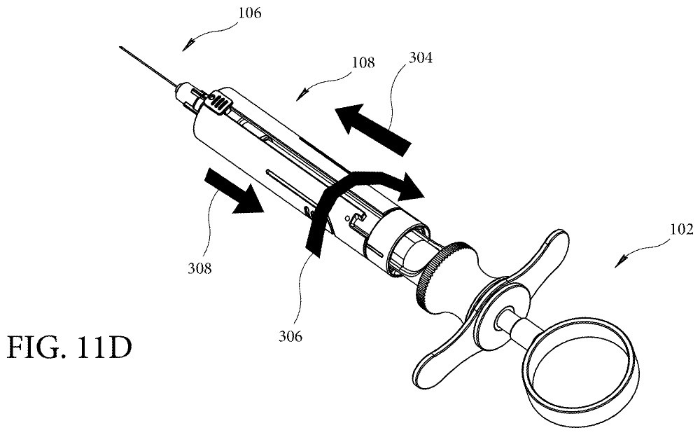

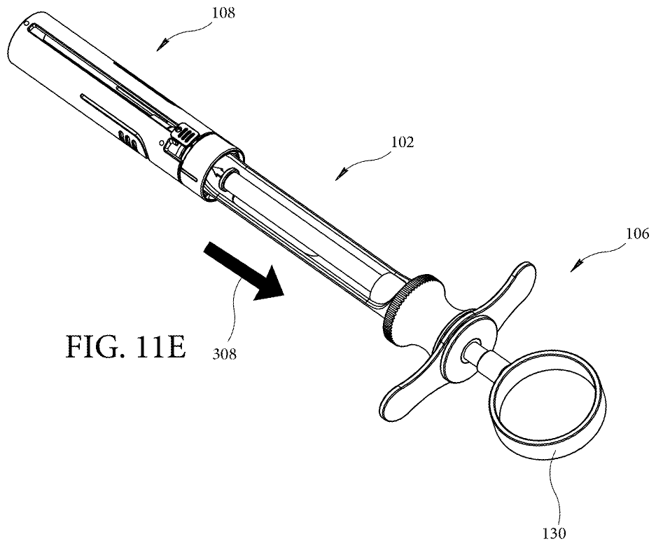

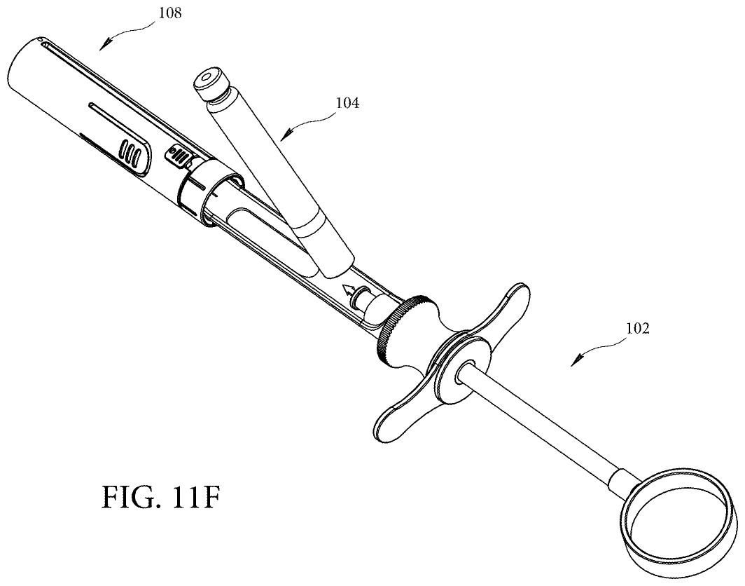

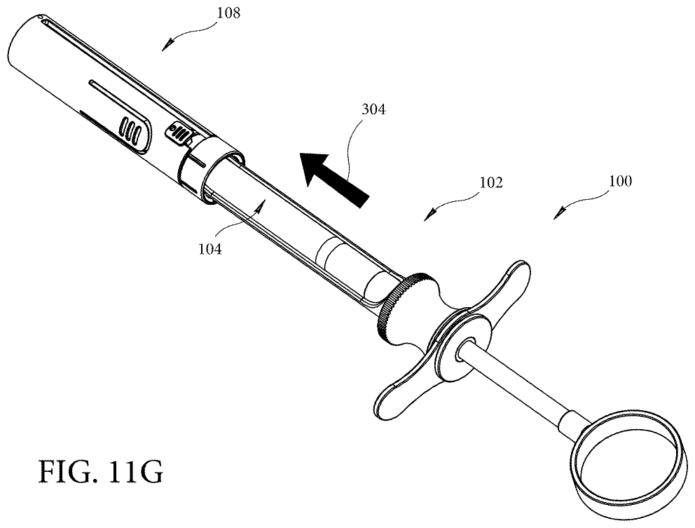

The safety device 108 may be mounted or attached to the syringe 102 and the needle assembly 106 may be mounted or attached to the syringe 102 before or after the carpule 104 is loaded into the syringe 102 as discussed above. Further details of at least one embodiment of a method for installing various structures, features, and components on or to the syringe 102 will be discussed with respect to FIGS. 10 and 11A-11G of the present disclosure later herein.

FIG. 2A is a perspective view of the injection system 100 with the carpule 104 present within the cavity 138 of the syringe 102, the safety device 108 mounted or attached to an exterior of the syringe 108, and the needle assembly 106 mounted or attached to the injection system 100. As shown in FIG. 2A, the cylindrical body 118 is in a retracted position such that the injection tip 112 of the needle assembly 106 is exposed, for example, when a medical practitioner or user is injecting a patient with a medication present within the carpule 104. When the cylindrical body 118 is in the retracted position as shown in FIG. 2A, the detent balls 129a, 129b are present within the detent openings 127a, 127b such that the cylindrical body 118 is temporarily locked into the retracted position. In other words, the retracted position may be referred to as a locked and retracted position of the cylindrical body 118 that is opposite to the locked and extended position of the cylindrical body 118. The internal base member 124 is at least partially covered by the needle hub 114 of the needle assembly 106, the internal base member 124, which may be a flange structure, is sandwiched between the needle hub 114 of the needle assembly 106 and the exterior surface 135 of the syringe 102 such that the internal base member 124 is stationary relative to the syringe 102 and the needle assembly 106. The needle hub 114 may be threaded (not shown) to threadedly engage with the threaded structure 134 of the syringe 102 such that the needle hub 114 may rotatedly thread onto the threaded structure 134 to mount the needle assembly 106 onto the syringe 102.

Alternatively, in some embodiments, the internal base member 124 may not be sandwiched between needle hub 114 of the needle assembly 106 and the exterior surface 135 of the syringe 102. Instead, the internal base member 124 may include a threaded portion that is configured to threadedly engage with a threaded portion on the syringe 102. The threaded portion may be different from the threaded structure 134 of the syringe 102. When the internal base member 124 includes the threaded portion, the threaded portion of the internal base member 124 is threaded onto the threaded portion of the syringe 102 such that the internal base member 124 is held stationary relative to the syringe 102.

Alternatively, in some embodiments, the internal base member 124 may not be sandwiched between the needle hub 114 of the needle assembly 106 and the exterior surface 135 of the syringe 102. Instead, the internal base member 124 may include a clamp structure that clamps down onto an exterior of the syringe 102 such that the internal base member 124 is held stationary relative to the syringe 102. For example, the clamp structure may be the same or similar to an alligator clip structure or some other type of clamp structure.

In some alternative embodiments, the internal base member 124 may include features and structures that may be the same or similar to a compression style fitting, which may be similar in structure and configuration to a compression style fitting utilized within plumbing scenarios. For example, the internal base member 124 may include a sleeve, a first threaded component, and a second threaded component. The sleeve may be slid onto the syringe 102 and the first threaded component may be positioned to overlap the sleeve. Once the sleeve and the first threaded component are positioned, the second threaded component is threadedly engaged with the first threaded component such that the first threaded component and the sleeve are compressed inward towards the syringe 102 by the second threaded component such that the sleeve, the first threaded component, and the second threaded component are held onto the syringe 102 by compression forces generated by threadedly engaging the second component with the first component.

In some alternative embodiments, the internal base member 124 may include a collet-style interface structure. For example, the internal base member 124 may be placed on or around the syringe 102 and the collect style-interface structure may be tightened such that flexure structures of the collect-style interface structure come into contact with the syringe 102. Once the collet-style interface structure is tightened enough, the internal base member 124 is held onto the syringe 102 by the flexure structures of the collet-style interface structure. In other words, respective ends of the flexure structures of the collet-style interface structure may come into contact with an exterior surface of the syringe 102 such that the flexure structures hold the internal base member 124 onto the syringe 102.

In some alternative embodiments, the internal base member 124 may slide over an end of the syringe 102 and include one or more snap fit structures that snap onto the syringe 102. For example, in some embodiments, each one of the one or more the snap fit structures may include a hook that wraps around an edge of the opening 136 or cavity 138 such that each one of the hooks extends into the opening 136. The hooks of the one or more snap fit structures result in the internal base member 124 being held onto the syringe 102. In some other embodiments, one of the hooks of the one or more snap fit structures of the internal base member 124 may hook into an opening on an opposite side of the syringe 102 relative to the opening 136 as shown in FIG. 1.

In some alternative embodiments, the internal base member 124 may be integral to the syringe 102 at an end of the syringe 102 similar to the end of the syringe 102 at which the threaded structure 134 is present as shown in FIG. 1. For example, in some embodiments, the internal base member 124 may be similar to the internal base member 124 as shown in FIG. 1 but instead is integral to the syringe 102 itself such that the internal base member 124 and the syringe 102 are made of a single, continuous material. This single, continuous material, for example, may be a medical grade stainless steel material. In other words, the internal base member 124 may be made of a single, continuous material along with the syringe 102 such that the internal base member 124 is permanently integral to the syringe 102. In some embodiments, the internal base member 124 may be permanently coupled to the syringe 102 by some other technique such as a welding technique or some other permanent coupling technique.

In view of these various embodiments of the internal base member 124, it will be readily appreciated that these various embodiments of the internal base member 124 may be mounted to, coupled to, or integral to the syringe 102 in various types of manners utilizing various types of mounting structures, features, or coupling techniques. In view of the above discussion with respect to the various types of mounting structures, features, and coupling techniques that may be utilized to position the internal base member 124 on the syringe 102, the various types of mounting structures, features, or coupling techniques may be utilized either alone or in combination with each other to removably mount the internal base member 124 to the syringe 102.

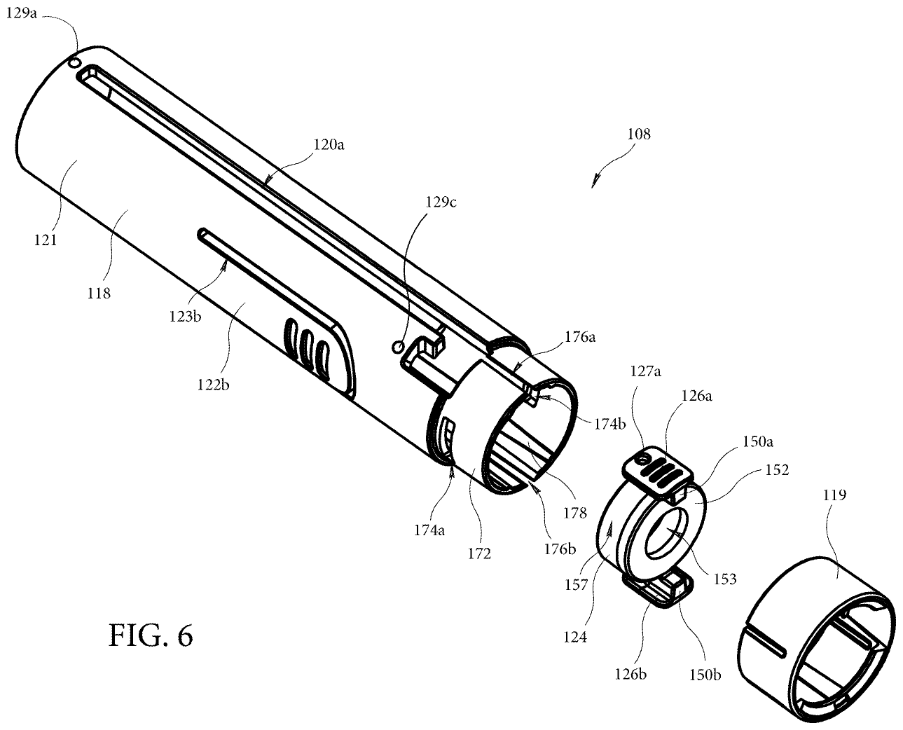

In view of the above discussion, the internal base member 124 is held stationary relative to the syringe 102 such that the cylindrical body 118 may move between the retracted position, the extended position, and the locked and extended position. For examples, surfaces of the opposing pathway slots 120a, 120b slide along opposing extensions 150a, 150b (see FIGS. 6 and 7A-7C of the present disclosure) of the internal base member 124 to move the cylindrical body 118 between the retracted position, the extended position, and the locked and extended position, respectively, since the internal base member 124 is held in a stationary position relative to the syringe 102 while the cylindrical body 118 is moved between the retracted position, the extended position, and the locked and extended position, respectively. As shown, the opposing pathway slots 120a, 120b are spaced apart from each other by an angle of 180-degrees relative to the cylindrical body 118.

While the first and second pathway slots 120a, 120b of the present disclosure are shown as being spaced apart by 180-degrees relative to each other and about the cylindrical body 118, in some alternative embodiments, the cylindrical body 118 may include only one of the first and second pathway slots 120a, 120b, include three pathway slots, include four pathway slots, or may include some other number of pathway slots. For example, when there is a third pathway slot, each one of the three pathway slots may be spaced apart from the other two pathway slots by an angle of 120-degrees, respectively, about the cylindrical body 118. Alternatively, when there are four pathway slots, each one of the four pathway slots may be spaced apart from two adjacent ones of the four pathway slots by an angle of 90-degrees, respectively, about the cylindrical body 118.

While the carpule tip 116 is not readily visible in FIG. 2A, the needle 110 pierces and extends through the diaphragm 144 such that the carpule tip 116 of the needle 110 is present within the medication cavity 146 of the carpule 104. The carpule tip 116 is within a medication present within the medication cavity 146 of the carpule 104 such that the medication may pass into the carpule tip 116, through the needle 110, and out the injection tip 112 when injecting a patient with the medication present within the medication cavity 146 of the carpule 104. For example, when the ring 130 of the syringe 102 is pushed by a medical practitioner or user of the injection system 100, the piston rod 128 moves towards the threaded structure 134 of the syringe 102 and the harpoon structure 132, which is engaged with the plunger 142, moves with the piston rod 128. This movement of the harpoon structure 132 along with the piston rod 128 moves the plunger 142 of the carpule 104 towards the stopper 140 of the carpule 104 such that the medication within the medication cavity 146 enters the carpule tip 116, passes through the needle 110, and exits through the injection tip 112 when injecting a patient with the medication within the medication cavity 146 of the carpule 104.

FIG. 2B is a perspective view of the injection system 100 as shown in FIG. 2A. However, unlike FIG. 2A in which the cylindrical body 118 is in a retracted position, the cylindrical body 118 is in an extended position. The cylindrical body 118 may be moved from the retracted position to the extended position by applying enough pressure to overcome interlocking forces between the detent openings 127a, 127b of the tab elements 126a, 126b and two of the detent balls 129a, 129b, respectively. Once interlocking forces between the detent openings 127a, 127b of the tab elements 126a, 126b and two of the detent balls 129a, 129b are overcome, the pathway slots 120a, 120b provide a degree of freedom such that the cylindrical body 118 may be moved from the retracted position as shown in FIG. 2A to the extended position as shown in FIG. 2B. In other words, since the internal base member 124 is held in place by being sandwiched between the needle hub 114 and the exterior surface 135, the tab elements 126a, 126b of the internal base member 124 are held in place as well while the pathway slots 120a, 120b provide the degree of freedom for the cylindrical body 118 to move from the retracted position to the extended position and vice versa.

When the cylindrical body 118 is in the extended position the injection tip 112 of the needle 110 is completely and fully covered by the cylindrical body 118 such that the injection tip 112 is not readily accessible. For example, the cylindrical body 118 completely and fully covers the injection tip 112 such that even if a medical practitioner or user of the injection system pressed a finger against an opening 148 of the cylindrical body 118, the finger could not reach the injection tip 112 of the needle 110 to avoid a needlestick injury. In other words, the opening 148 is structured to be small enough such that a finger cannot travel far enough into the cylindrical body 118 to reach the injection tip 112 of the needle 110. The safety device 108 is structured such that the end of the safety device 108 extends far enough past the injection tip 112 of the needle 110 of the needle assembly 106 when in the locked and extended position as shown in FIG. 2C such that a finger cannot travel far enough into the cylindrical body 118 to reach the injection tip 112 of the needle 110. For example, in at least one embodiment, the needle 110 may have a length that extends from the carpule tip 116 to the injection tip 112 that is substantially equal to 1.375-inches (in), which is approximately equal to 35-millimeters (mm), and the end of the safety device 108 extends beyond the injection tip 112 of the needle 110 when in the locked and extended position as shown in FIG. 2C. The cylindrical body 118 may be referred to as a sheath, a guard, or some other type of reference to the cylindrical body 118 that is configured to limit, prevent, stop, or block access to the injection tip 112 when in the extended position as shown in FIG. 2B and in the extended and locked position as shown in FIG. 2C.

In some alternative embodiments, the cylindrical body 118 may include one or more flexible structures or fingers that cover the opening 148 when the cylindrical body 118 is in the extended position as shown in FIG. 2C such that the injection tip 112 of the needle 110 is covered by the flexible structures or fingers of the cylindrical body 118. The one or more flexible structures or fingers may be integral to the cylindrical body 118 and may be present at the end of the cylindrical body 118 adjacent to the opening 148. For example, the flexible structures or fingers may be tapered inward such as to cover the opening 148 when the cylindrical body 118 is in the extended position (see, e.g., FIG. 2C), and there may be respective slots that are between each one of the flexible structures or fingers that allow the flexible structures and fingers to elastically deform. These slots allow the flexible structures or fingers to deform when the cylindrical body 118 is moved from the extended position to the retracted position (see, e.g., FIG. 2A) such that the flexible structures or fingers flex and elastically deform outward exposing the opening 148 and the injection tip 112 of the needle 110 of the needle assembly 106. The flexible structures or fingers may flex and elastically deform outward as the flexible structure or fingers pass along and by the needle hub 114 of the needle assembly 106. For example, the respective ends of the flexible structures or fingers are pushed outward as the respective ends of the flexible structures or fingers slide along an exterior surface of the needle hub 114 of the needle assembly 106. The flexible structures or fingers may act as a physical barrier to prevent the medical practitioner from incurring a needlestick injury when the cylindrical body 118 is in the extended position.

FIG. 2C is a perspective view of the injection system 100 as shown in FIGS. 2A and 2B. However, unlike FIGS. 2A and 2B, the cylindrical body is in a locked position in which the cylindrical body 118 remains extended (e.g., the locked and extended position) such that the injection tip 112 is not readily accessible as discussed earlier with respect to FIG. 2B. When in the locked position, the detent openings 127a, 127b of the tab elements 126a, 126b receive the detent balls 129c, 129d, respectively, such that the cylindrical body is temporarily locked into the locked position as shown in FIG. 2C. In other words, the detent balls 129c, 129d, respectively, are inserted into the detent openings 127a, 127b of the tab elements 126a, 126b. The cylindrical body 118 may be moved from the extended position as shown in FIG. 2B to the locked position as shown in FIG. 2C by rotating the cylindrical body 118 such that surfaces delimiting the pathway slots 120a, 120b slide along the tab elements 126a, 126b, and then moving the cylindrical body 118 slightly towards the ring 130 of the syringe such that the detent balls 129c, 129d are inserted into two of the detent recesses 127a, 127b, respectively. Once the detent balls 129c, 129d are inserted into the detent openings 127a, 127b, the cylindrical body is temporarily locked into the locked position as shown in FIG. 2C.

In view of the above discussion of FIGS. 2A, 2B, and 2C, the cylindrical body 118 may be readily moved from the retracted position to the extended position and then to the locked position. Alternatively, the cylindrical body 118 may be readily moved from the locked position to the extended position and then to the locked position. For example, a medical practitioner or user of the injection system 100 applies enough force to overcome interlocking forces between the detent balls 129c, 129d and the detent openings 127a, 127b, the cylindrical body 118 may be moved slightly away from the ring 130 of the syringe 102 and then rotated such that surfaces delimiting the pathway slots 120a, 120b slide along the tab elements 126a, 126b such that the cylindrical body 118 moves from the locked position as shown in FIG. 2C to the extended position as shown in FIG. 2B. After the cylindrical body 118 is in the extended position as shown in FIG. 2B, the cylindrical body 118 is moved from the extended position as shown in FIG. 2B to the retracted position as shown in FIG. 2A.

In view of the above discussion with respect to FIGS. 2A-2C, in some alternative embodiments, at least some or all of the detent balls 129a, 129b, 129c, 129d and at least some or all of the detent openings 127a, 127b may be replaced with different types of friction fit structures that hold the cylindrical body 118 in the retracted position as shown in FIG. 2A as well as in the extended position as shown in FIG. 2C. For example, similar to the detent balls 129a, 129b, 129c, 129d and the detent openings 127a, 127b, a first friction fit structure at an end of the cylindrical body 118 closest to the ring structure 130 may frictionally hold the cylindrical body 118 in the extended position as shown in FIG. 2C, and a second friction fit structure at an end of the cylindrical body 118 farthest from the ring structure 130 may frictionally hold the cylindrical body in the retracted position as shown in FIG. 2A. When enough force is applied to the cylindrical body 118, a friction force when in the retracted position may be overcome such that the cylindrical body 118 may be moved to the extended position and vice versa. The first friction fit structure may be a first tapered structure and the second friction fit structure may be a second tapered structure. In yet some further alternative embodiments, only one of the first or second friction fit structures may be present. In some embodiments, either one of or both of the first and second tapered structures may be conical.

In view of the above discussion with respect to FIGS. 2A-2C, in some alternative embodiments, at least some or all of the detent balls 129a, 129b, 129c, 129d and at least some or all of the detent openings 127a, 127b may be replaced with magnets that hold the cylindrical body 118 in the retracted position as shown in FIG. 2A as well as in the extended position as shown in FIG. 2C. For example, the detent balls 129a, 129b, 129c, 129d may be replaced with first magnets and the detent openings 127a, 127b may be replaced with second magnets. For example, similar to the detent balls 129a, 129b, 129c, 129d and the detent openings 127a, 127b, when the cylindrical body 118 is in the retracted position as shown in FIG. 2A, each one of the second magnets is attracted to a corresponding one of a first pair of the first magnets such that the second magnets are attracted to the first pair of the first magnets and the cylindrical body 118 is magnetically held in the retracted position by the attraction of the second magnets to the first pair of first magnets. When the cylindrical body is in the extended position as shown in FIG. 2C, each one of the second magnets is attracted to a corresponding one of a second pair of the first magnets such that the second magnets are attracted to the second pair of the first magnets and the cylindrical body 118 is magnetically held in the extended position by the attraction of the second magnets to the second pair of first magnets. When enough force is applied to the cylindrical body 118, a magnetic force when in the retracted position may be overcome such that the cylindrical body 118 may be moved to the extended position and vice versa. The first magnets may be in the same or similar position as the detent balls 129a, 129b, 129c, 129d, and the second magnets may be in the same or similar position as the detent openings 127a, 127b.

In some alternative embodiments, the safety device 108 may include a threaded element or structure (not shown) that allows the cylindrical body 118 to move between the retracted position (see, e.g., FIG. 2A) and the extended position (see, e.g., FIG. 2C). For example, the threaded element or structure may be rotated by an amount to move the cylindrical body 118 from the retracted position to the extended position and vice versa. For example, the amount of the threaded element or structure may be a quarter (¼) turn, a half (½) turn, a full turn, or some other amount of a turn to move the cylindrical body 118 from the retracted position to the extended position and vice versa.

In some alternative embodiments, the safety device 108 may include a latch structure or mechanism that may replace the detent balls 129a, 129b, 129c, 129d and the detent openings 127a, 127b. For example, the latch structure or mechanism may include a flexible portion about which a fulcrum is positioned. A first end of the flexible portion may engage with a first reception structure when the cylindrical body 118 is in the retracted position (see, e.g., FIG. 2A) such that the cylindrical body 118 may be locked in the retracted position, and a second end of the flexible portion may engage with a second reception structure when the cylindrical body 118 is in the extended position (see, e.g., FIG. 2C) such that the cylindrical body 118 may be locked in the extended position. When enough force is applied to the cylindrical body 118 or the flexible portion, an engagement force between the first end of the flexible portion engaged with the first reception structure is overcome such that the cylindrical body 118 may be moved from the retracted position to the extended position. When enough force is applied to the cylindrical body 118 or the flexible portion, an engagement force between the second end of the flexible portion engaged with the second reception structure is overcome such that the cylindrical body 118 may be moved from the extended position to the retracted position.

In some embodiments, the safety device 108 may include one or more springs that may replace the detent balls 129a, 129b, 129c, 129d and the detent openings 127a, 127b. The one or more springs may apply enough force to the cylindrical body 118 when in the retracted position (see, e.g., FIG. 2A) to lock the cylindrical body 118 in the retracted position. The one or more springs may apply enough force to the cylindrical body 118 when in the extended position (see, e.g., FIG. 2C) to lock the cylindrical body in the extended position. When enough force is applied to the cylindrical body 118 to overcome the forces of the one or more springs, the cylindrical body may be moved from the retracted position to the extended position or vice versa.

In view of the above discussion with respect to replacing the detent balls 129a, 129b, 129c, 129d and the detent openings 127a, 127b, it will be readily appreciated that various types of locking features, structures, and components may be utilized to hold the cylindrical body 118 statically in the retracted position and the extended position until enough force is applied to overcome the interlocking forces to move the cylindrical body 118 from the retracted position to the extended position and vice versa. In view of the above discussion with respect to the various types of locking features, structures, and components to hold the cylindrical body 118 in the retracted and extended positions as shown in FIGS. 2A and 2C, these various types of locking features, structures, and components may be utilized either alone or in combination with each other to hold the cylindrical body 118 statically in the retracted position as shown in FIG. 2A or in the extended position as shown in FIG. 2C.

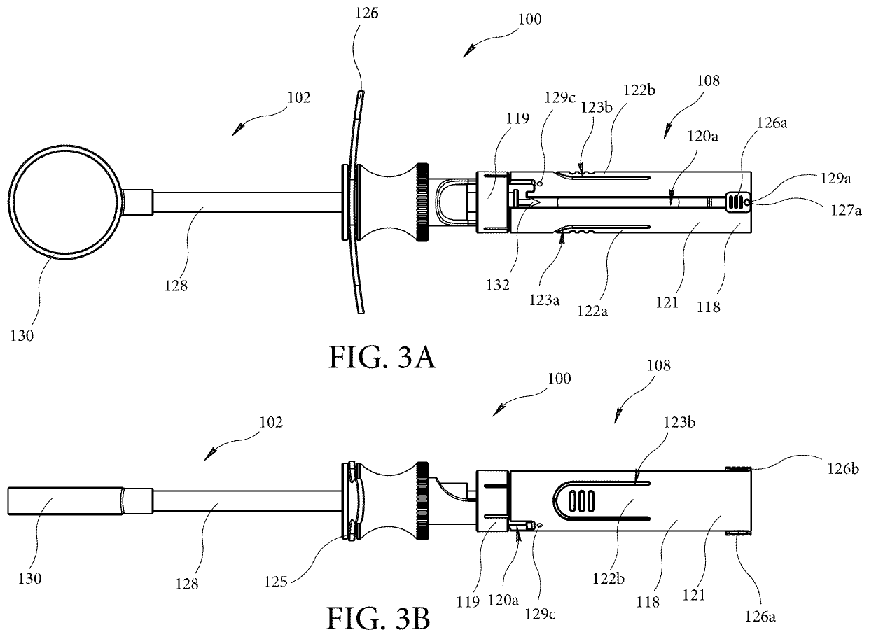

FIG. 3A is a side view of the injection system 100. As may be readily seen in FIG. 3A, the first pathway slot 120a has a fish hook-like shape or a J-shape along which the first tab element 126a is present. For example, the J-shape includes a first horizontal portion, a second horizontal portion, and a third vertical portion transverse to the first and second horizontal portions based on the orientation of the injection system 100 as shown in FIG. 3A. The third vertical portion extends from the first horizontal portion to the second horizontal portion as shown in FIG. 3A. As shown in FIG. 3A, the detent ball 129a is adjacent to an end of the first horizontal portion of the first pathway slot 120a based on the orientation as shown in FIG. 3A, and the detent ball 129c is adjacent to an end of the second horizontal portion of the first pathway slot 120a based on the orientation as shown in FIG. 3A. As shown in FIG. 3A, the detent ball 129a is in the detent opening 127a. While not readily visible in FIG. 3A, the second pathway slot 120b is the same or similar to the first pathway slot 120a and the second tab element 126b is the same or similar to the first tab element 126a, and the positional relationship between the second pathway slot 120b and the detent balls 129b, 129d, respectively, is the same or similar as the positional relationship between the first pathway slot 120a and the detent balls 129a, 129c, respectively. However, the second pathway slot 120b and the second tab element 126b are on an opposite side of the cylindrical body 118 relative to the side of the cylindrical body 118 as shown in FIG. 3A at which the first pathway slot 120a and the first tab element 126a are present. In other words, the second pathway slot 120b and the second tab element 126b are mirror images to the first pathway slot 120a and the first tab element 126a. As shown in FIG. 3A, the needle assembly 106 is not present and the safety device 108 is in the retracted position.

In some alternative embodiments, the first pathway slot 120a and the second pathway slot 120b may be horizontal slots such that the second horizontal portion and the third vertical portion of the first and second pathway slots 120a, 120b are not present. In other words, in some alternative embodiments, the first and second pathway slots 120a, 120b include only the first horizontal portion, respectively. When the first pathway slot 120a includes only the first horizontal portion, respectively, the detent ball 129a is at a first end of the first pathway slot 120a and the detent ball 129c is at a second end of the first pathway 120a opposite to the first end of the first pathway slot 120a. When the second pathway slot 120b includes only the first horizontal portion, respectively, the detent ball 129b is at a first end of the second pathway slot 120b and the detent ball 129d is at a second end of the second pathway slot 120b opposite to the first end of the second pathway slot 120b.

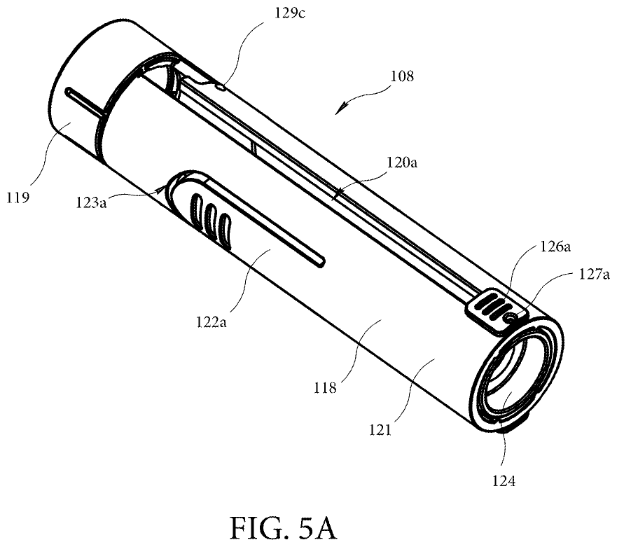

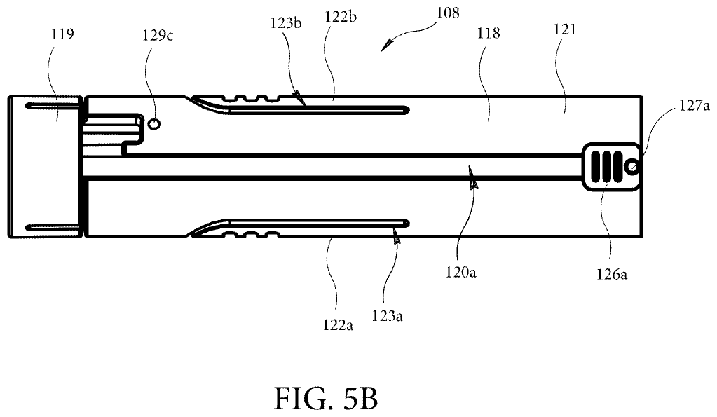

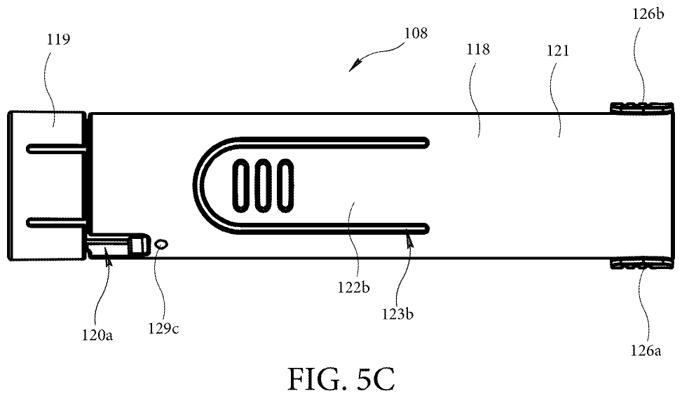

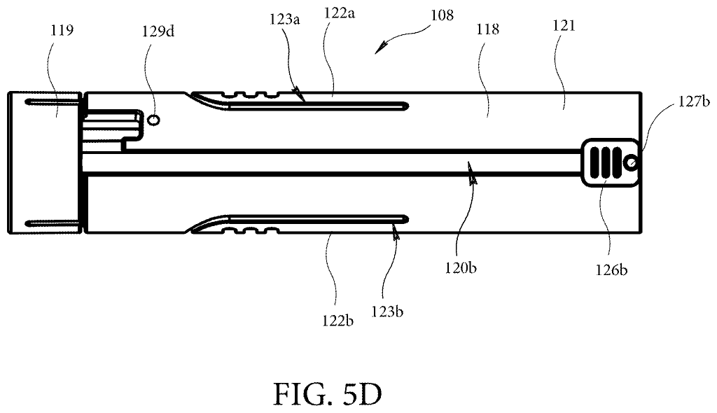

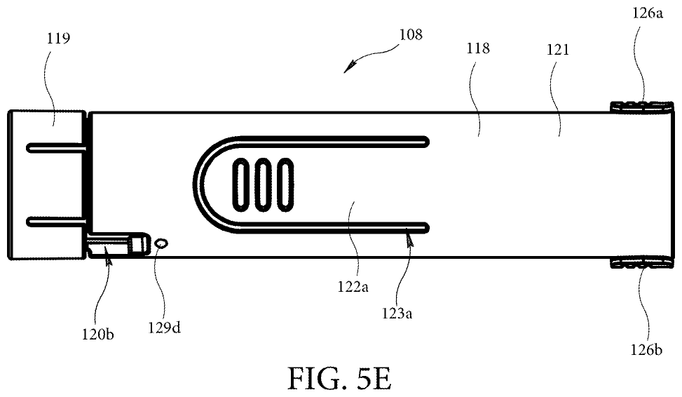

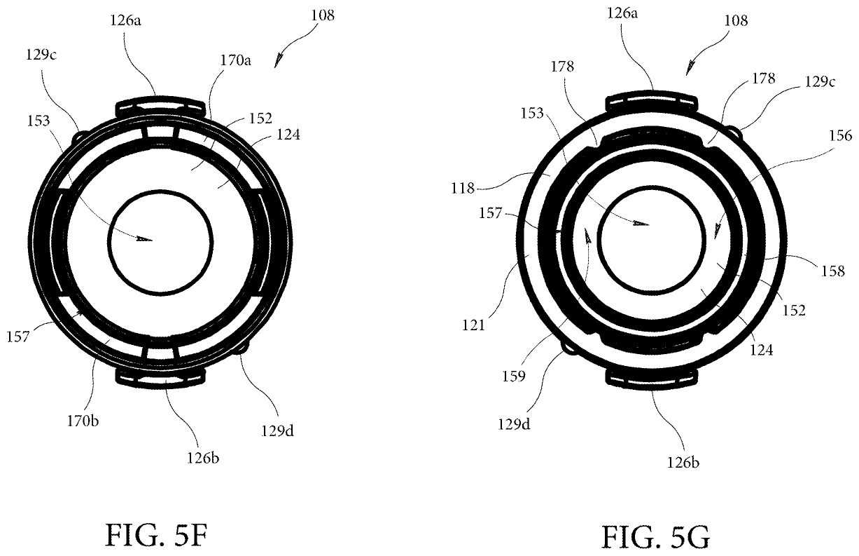

FIG. 3B is a side view of the injection system 100. As may be not readily seen in FIG. 3B, the first slot 123a has a U-shape and is directly adjacent to the first flexible and elastically deformable grip element 122a (see FIG. 5E for U-shape of the first slot 123a). The first slot 123a is configured to, during operation, allow the first flexible and elastically deformable grip element 122a to be elastically deformed inward towards the central axis of the cylindrical body 118. As may be readily visible in FIG. 3B, the second slot 123b has a U-shape and is directly adjacent to the second flexible and elastically deformable grip element 122b. The second slot 123b is configured to, during operation, allow the second flexible and elastically deformable grip element 122b to be elastically deformed inward towards the central axis of the cylindrical body 118. As shown in FIG. 3B, the needle assembly 106 is not present on the syringe 102 and the safety device 108 is in the retracted position. In some alternative embodiments, the first and second slots 123a, 123b may have an L-shape instead of a U-shape. In other words, the first and second slots 123a, 123b may be selected from any type of shapes that may allow the first and second flexible and elastically deformable grip elements 122a, 122b to be elastically deformed towards the central axis of the cylindrical body 118, respectively.

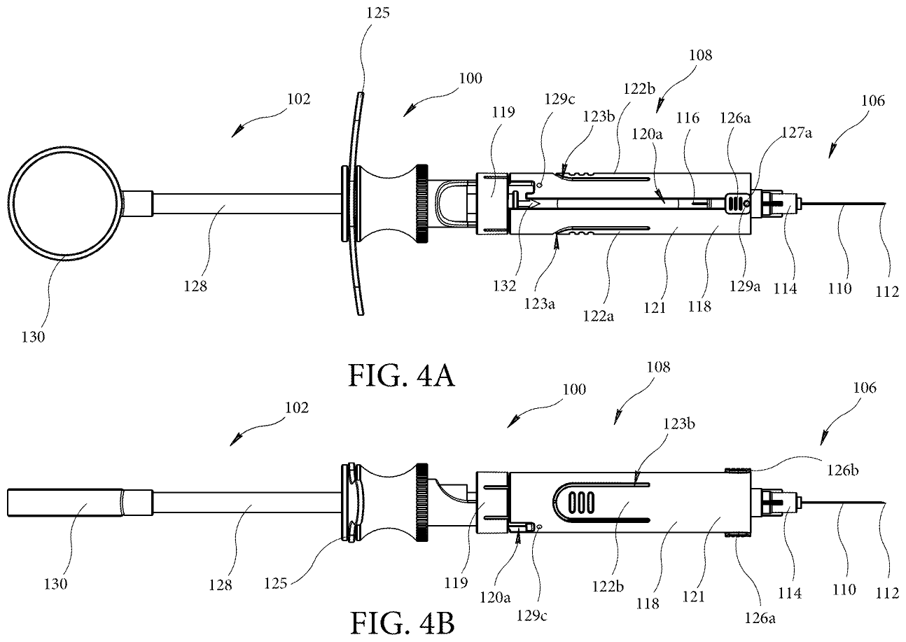

FIG. 4A is a side view of the injection system 100 similar to the side view of the injection system 100 as shown in FIG. 3A. However, unlike FIG. 3A, the needle assembly 106 is present such that the needle hub 114 of the needle assembly 106 is threadedly engaged with the threaded structure 134 of the syringe 102.

FIG. 4B is a side view of the injection system 100 similar to the side view of the injection system 100 as shown in FIG. 3B. However, unlike FIG. 3B, the needle assembly 106 is present such that the needle hub 114 of the needle assembly 106 is threadedly engaged with the threaded structure of the syringe 102.

FIG. 5A is a perspective view of the safety device 108. FIG. 5B is a right side view of the safety device 108. FIG. 5C is a front side view of the safety device 108. FIG. 5D is a left side view of the safety device 108. FIG. 5E is a rear side view of the safety device 108. FIG. 5F is a bottom side view of the safety device 108. FIG. 5G is a top side view of the safety device 108.

As shown in FIG. 5F, the internal base member 124 includes opposing extensions 150a, 150b (see FIGS. 6 and 7A-7C) that extend from a flange 152 of the internal base member 124 to the tab elements 126a, 126b. The flange 152 is structured to be sandwiched between the needle hub 114 of the needle assembly 106 and the exterior surface 135 of the syringe 102. The first extension 150a extends to the first tab element 126a and extends through the first pathway slot 120a, and the second extension 150b extends to the second tab element 126b and extends through the second slot 120b. The opposing extensions 150a, 150b are within the opposing pathway slots 120a, 120b such that surfaces defining or delimiting the pathway slots 120a, 120b slide along the opposing extensions 150a, 150b when moving the cylindrical body 118 between the retracted position, the extended position, and the locked and extended position, respectively.

While the first and second pathway slots 120a, 120b are shown extending entirely through the cylindrical body 118 in FIGS. 5A, 5B, and 5D, in some alternative embodiments, the first and second pathway slots 120a, 120b may not extend entirely through the cylindrical body 118. Instead, in some alternative embodiments, the first and second pathway slots 120a, 120b may be replaced with internal pathway recesses or tracks that are present at an inner surface of the cylindrical body 118. When the first and second pathway slots 120a, 120b are replaced with internal pathway recesses or tracks, the internal base member 124 may include structures similar to the extensions 150a, 150b that extend into the internal pathway recesses or tracks such that these structures may slide along the internal pathway recesses or tracks, respectively. This sliding of the structures similar to the extensions 150a, 150b along the internal pathway recesses or tracks may be similar to the first and second pathway slots 120a, 120b sliding along the extensions 150a, 150b of the internal base member 124 such that the cylindrical body 118 may be moved from the retracted position to the extended position and vice versa.

In yet some other alternative embodiments, the internal base member 124 may include the extensions 150a, 150b but not include the tab structures 126a, 126b such that the extensions 150a, 150b only partially extend into the first and second pathway slots 120a, 120b. In other words, in these other alternative embodiments, the first and second extensions 150a, 150b may terminate at respective ends of the first and second extensions 150a, 150b present within the first and second pathway slots 120a, 120b such that the extensions 150a, 150b do not extend entirely through the first and second pathway slots 120a, 120b.

An opening 153 extends through the flange 152 of the internal base member 124. The opening 153 is structured to allow the carpule tip 116 of the needle 110 of the needle assembly 106 to pass through the flange 152 and to pass through the threaded structure 134 of the syringe 102 such that the carpule tip 116 of the needle 110 is inserted into the cavity 138 of the syringe 102. The opening 153 is structured and has a diameter larger than a diameter of the threaded structure 134 of the syringe 102 such that the threaded structure 134 may pass through the opening 153 such that the needle hub 114 may be threaded onto the threaded structure 134 when the safety device 108 is mounted to the syringe 102 and the needle assembly 106 is mounted to the syringe 102.

As shown in FIG. 5G, the cylindrical body 118 includes one or more ribs or projections 178 that protrude from an interior surface 155 (see FIGS. 9A-9C of the present disclosure) of the cylindrical body 118 towards the central axis of the cylindrical body 118. The one or more ribs or projections 178 may extend towards an exterior surface 157 (see FIGS. 6 and 7A-7C of the present disclosure) of the internal base member 124. However, the one or more ribs or projections 178 along the interior surface 155 of the cylindrical body 118 are spaced apart from the exterior surface 157 of the internal base member 124 such that the cylindrical body 118 may slide along the opposing extensions 150a, 150b between the retracted position, the extended position, and the locked and extended position, respectively.

FIG. 6 is an exploded view of the safety device 108. As shown in FIG. 6, the safety device includes the first portion 119, the second portion 121 that is coupled to the first portion 119, and the internal base member 124 that may be present within the first portion 119, the second portion 121, or both depending on a position of the first and second portions 119, 121, respectively, relative to the internal base member 124.

FIG. 7A is a perspective view of the internal base member 124. As shown in FIG. 7A, the internal base member 124 includes a recess 156 that is aligned with the opening 153 that extends through the flange 152. A peripheral portion 158 extends around the recess 156. The recess 156 is configured to receive the needle hub 114 of the needle assembly 106. For example, a surface 159 may abut a lower surface of the needle hub 114 of the needle assembly 106 when the safety device 108 and the needle assembly 106 are mounted to the syringe 102.

In some alternative embodiments, the recess 156 and the peripheral portion 158 may not be present such that only the flange 152 is present. In other words, in these alternative embodiments, the flange 152 may be a flat disc or a flat flange structure.

FIG. 7B is a top plan view of the internal base member 124. FIG. 7C is a bottom plan view of the internal base member 124. As shown in FIG. 7C, a surface 160 of the internal base member 124 may abut the exterior surface 135 of the syringe 102 when the safety device 108 is mounted to the syringe 102.

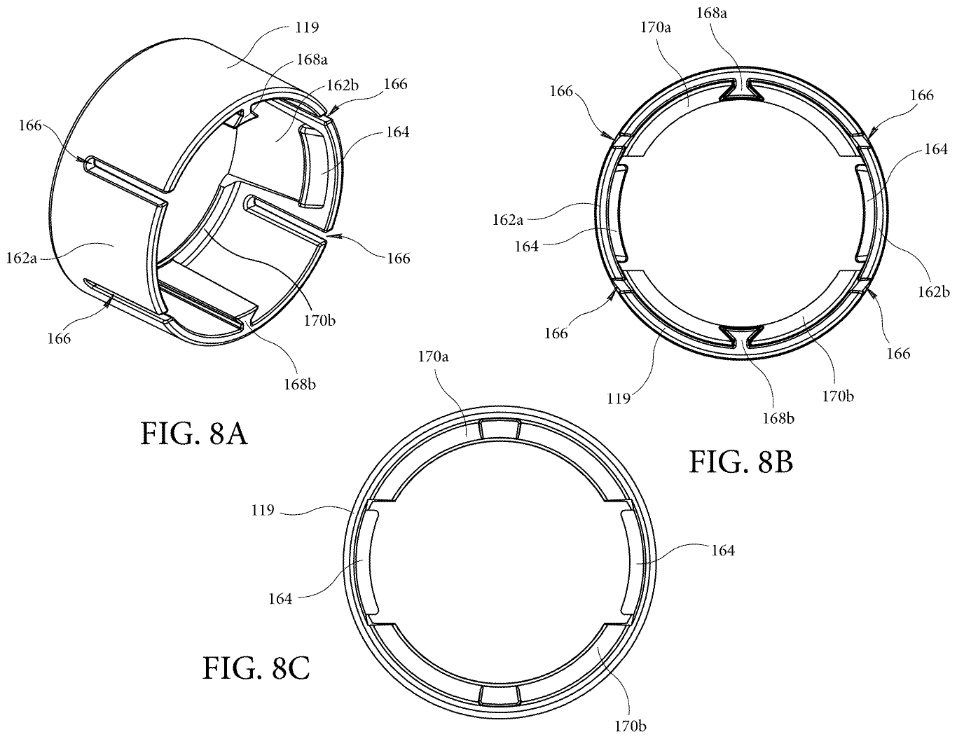

FIG. 8A is a perspective view of the first portion 119 of the cylindrical body 118. As shown in FIG. 8A, the first portion 119 includes opposing snap fit structures 162a, 162b each of which includes a protrusion 164. Flexure slots 166 are adjacent to the opposing snap fit structures 162a, 162b such that the opposing snap fit structures 162a, 162b may flex and elastically deform when snapping the first portion 119 onto the second portion 121 to define and form the cylindrical body 118 of the safety device 108. The first portion 119 further includes opposing guide structures 168a, 168b that direct the first portion 119 when coupling the first portion 119 to the second portion 121 such that the first portion 119 properly snaps into place in an appropriate orientation relative to the second portion 121. These opposing guide structures 168a, 168b may be structured and configured to prevent the second portion 121 of the cylindrical body 118 from deflecting inward. The opposing guide structures 168a, 168b are flared outward such that the opposing guide structures 168a, 168b include a wide portion and a narrow portion that is less wide than the wide portion. The first portion 119 further includes opposing lip structures 170a, 170b that are configured to abut the second portion 121 when coupling the first portion 119 to the second portion 121 such that the opposing lip structures 170a, 170b act as stoppers to assist in stopping or preventing the first portion 119 from sliding farther onto the second portion 121. The first portion 119 may be referred to as a ring structure, a stopper structure, or some other similar or like reference to the first portion 119. FIG. 8B is a top plan view of the first portion 119, and FIG. 8C is a bottom plan view of the first portion 119.

FIG. 9A is a perspective view of the second portion 121 of the cylindrical body 118 of the safety device 118. As shown in FIG. 9A, the second portion 121 includes a reception structure 172 with opposing snap fit slots 174a, 174b, opposing guide slots 176a, 176b, and the one or more ribs or projections 178. When coupling the first portion 119 to the second portion 121, the first portion 119 is slid onto the reception structure 172 of the second portion 121. As the first portion 119 is slid onto the second portion 121, the opposing guide structures 168a, 168b are inserted and slid into the opposing guide slots 176a, 176b. Once the first portion 119 is slid far enough onto the reception structure 172, the protrusions 164 of the opposing snap fit structures 162a, 162b reach the opposing snap fit slots 174a, 174b such that the protrusions 164 of the opposing snap fit structures 162a, 162b snap into the opposing snap fit slots 174a, 174b, respectively. This mechanically engages the protrusions 164 of the opposing snap fit structures 162a, 162b with the opposing snap fit slots 174a, 174b such that the first portion 119 is coupled to the second portion 121. The one or more ribs or projections 178 may be configured to act as spacers to space an interior surface of the second portion 121 from an exterior surface of the syringe 102 such that the cylindrical body 118 may be moved between the retracted position, the extended position, and the locked and extended position, respectively. For example, the one or more ribs or projections 178 may extend to and may abut the exterior surface of the syringe 102 such that the interior surface of the second portion 121 of the cylindrical body 118 from which the one or more ribs or projections 178 extend or project from is spaced apart from the exterior surface of the syringe 102 to provide enough clearance between the interior surface of the second portion 121 of the cylindrical body 118 and the exterior surface of the syringe 102. FIG. 9B is a top plan view of the second portion 121, and FIG. 9C is a bottom plan view of the second portion 121.

In some alternative embodiments, the one or more ribs or projections 178 may not be present. For example, the interior surface of the second portion 121 of the cylindrical body 118 may instead be a smooth, continuous surface. In some embodiments, when the interior surface is the smooth, continuous surface, the interior surface of the second portion 121 of the cylindrical body 118 may be directly adjacent to or abut the exterior surface of the syringe 102. In some embodiments, when the interior surface is the smooth, continuous surface, the interior surface of the second portion 121 of the cylindrical body 118 may be spaced apart from the exterior surface of the syringe 102.

In view of the above discussion, the internal base member 124 may be referred to as a third portion of the safety device 108. For example, as shown in FIG. 1 of the present disclosure, the first portion 119 and the second portion 121 define the cylindrical body 118 of the safety device 108 and the third portion 124 (e.g., the internal base member 124) is within the first and second portions 119, 121 such that the first and second portions 119, 121 have degrees of freedom due to the opposing pathway slots 120a, 120b relative to the third portion 124 (e.g., the internal base member 124). In other words, the opposing pathway slots 120a, 120b allow the first and second portions 119, 121 to move relative to the third portion 124 (e.g., the internal base member 124) between various positions of the first and second portions 119, 121, which define the cylindrical body 118 of the safety device 108, relative to the third portion 124, which is the internal base member 124 of the safety device 108.

In view of the above discussion, before the first portion 119 is coupled to the reception structure 172 of the second portion 121 of the safety device 108, the opposing extensions 150a, 150b, respectively, are moved through (e.g., slid through) the opposing guide slots 176a, 176b, respectively, such that the third portion 124 (e.g., internal base member 124) is present within the second portion 121 and the opposing extensions 150a, 150b are within the opposing pathway slots 120a, 120b, respectively. Once the third portion 124 (e.g., internal base member 124) is within the second portion 121, the first portion 119 is coupled to the reception structure 172 of the second portion 121

FIG. 10 is directed to a flowchart 200 of an embodiment of a method of preparing the injection system 100 to be utilized to inject a patient, which may be before, during, or after an operation, procedure, or surgery on the patient. The steps of FIG. 10 with respect to the embodiment of the method of preparing the injection system 100 will be discussed with respect to FIGS. 11A-11G. It will be readily appreciated that the steps as shown in the flowchart 200 may be reorganized or completed in a different order than as shown in FIG. 10.

In a first step 202 of the flowchart 200 illustrating the method, the safety device 108 is positioned on the syringe 102 such that the surface 160 is abutting the exterior surface 135 of the syringe 102. As shown in FIG. 11A, when positioning the safety device 108 onto in the first step 202, the cylindrical body 118 is in the retracted position and is locked in the retracted position by the detent balls 129a, 129b being present within the detent openings 127a, 127b, respectively. The syringe 102 is inserted into an opening (e.g., a cylindrical opening, recess, or cavity) defined by the first portion 119 of the cylindrical body 118, the second portion 121 of the cylindrical body 118, and the internal base member 124. Alternatively, the cylindrical body 118 may be in the locked and extended position (see FIG. 2C of the present disclosure) when positioning the safety device 108 on the syringe 102 and may then be moved from the locked and extended position to the retracted position. The syringe 102 is inserted into the safety device 108 such that the threaded structure 134 is inserted into the opening 153 of the internal base member 124 and passes through the opening 153 of the internal base member 124.

After the first step 202 in which the safety device 108 is positioned onto the syringe 102 by inserting the syringe 102 into the safety device 108, a second step 204 is performed in which the needle assembly 106 is mounted to the syringe 102 as shown in FIG. 11B. A carpule tip cap 300 is removed from the needle assembly 106 to expose the carpule tip 116 of the needle 110 of the needle assembly 106. The carpule tip cap 300 was previously removably on the needle hub 114. The needle assembly 106 is then mounted to the syringe 102 by threadedly engaging the needle hub 114 with the threaded structure 134 of the syringe 102. In other words, the needle hub 114 is screwed onto the threaded structure 134 of the syringe 102. The carpule tip 116 of the needle 110 is inserted through the threaded structure 134 of the syringe 102 such that the carpule tip 116 is present within the cavity 138 of the syringe 102.

After the second step 204 in which the needle hub 114 of the needle assembly 106 is threadedly engaged with the threaded structure 134, in a third step 206 an injection tip cap 302 is removed from the needle assembly 106 to expose the injection tip 112 of the needle 110 as shown in FIG. 11C. The injection tip cap 302 was previously removably on the needle hub 114.

After the third step 206 in which the injection tip cap 302 is removed from the needle assembly 106 to expose the injection tip 112 of the needle 110, in a fourth step 208 the cylindrical body 118 of the safety device 108 is moved from the retracted position to the locked and extended position (see FIG. 2C of the present disclosure). Moving the cylindrical body 118 to the locked and extended position, fully and completely covers the injection tip 112 of the needle such that a medical practitioner or user cannot poke or stick themselves with the injection tip 112 of the needle 110. This reduces the likelihood of a needlestick injury to the medical practitioner or the user due to miss-utilization or dropping of the injection system 100. For example, the medical practitioner or user applies enough force to remove the detent balls 129a, 129b from the detent openings 127a, 127b and then moves the cylindrical body 118 in a first direction represented by an arrow 304 by sliding surfaces defining or delimiting the opposing pathway slots 120a, 120b along the opposing extensions 150a, 150b integral to the interfacing tab elements 126a, 126b. Once the cylindrical body 118 is in the extended position as shown in FIG. 2B, the medical practitioner or the user rotates the cylindrical body 118 represented by an arrow 306. After rotating the cylindrical body 118, the medical practitioner or the user then moves the cylindrical body in a second direction represented by an arrow 308 such that the detent balls 129c, 129d are inserted into the detent opening 127a, 127b such that the cylindrical body 118 is in the locked and extended position as shown in FIG. 2C. This process may be reversed to move the cylindrical body from the locked and extended position as shown in FIG. 2C to the locked and retracted position as shown in FIG. 2A.

After the fourth step 208 in which the cylindrical body 118 of the safety device 108 is moved from the retracted position (see FIG. 2A of the present disclosure) to the locked and extended position (see FIG. 2C of the present disclosure), in a fifth step 210 the piston rod 128 is moved from a compressed state as shown in FIG. 11E to an extended position by actuating the ring 130 in the second direction represented by the arrow 308 as shown in FIG. 11F. For example, a medical practitioner or user may pull on the ring 130 such that the piston rod 128 is slid to the extended position (see FIGS. 1, 2A-2C, and 11F of the present disclosure).

After the fifth step 210 in which the piston rod 128 is moved to the extended position as shown in FIG. 11F, in a sixth step 212 the carpule 104 is inserted into or loaded into the cavity 138 of the syringe 102 through the opening 136 of the syringe 102. For example, the carpule 104 is inserted through the opening 136 into the cavity 138 of the syringe 102 as is laid within and supported within the cavity 138 of the syringe 102. The plunger 142 of the carpule 104 is closer to the harpoon structure 132 at the end of the piston rod 128 than the stopper 140 such that the plunger 142 faces the harpoon structure 132. The stopper 140 of the carpule 104 is closer to the carpule tip 116 of the needle 110 of the needle assembly 106 than the plunger 142 such that the diaphragm 144 of the stopper 140 faces the carpule tip 116 of the needle 110. When the carpule 104 is present within the cavity 138, the opening (not shown) of the plunger 142 that is structured to receive the harpoon structure 132 is aligned with the harpoon structure 132, and the diaphragm 144 of the stopper 140 is aligned with the carpule tip 116 of the needle 110.

After the sixth step 212 in which the carpule 104 is inserted or loaded into the cavity 138 of the syringe 102 through the opening 136 of the syringe 102, in a seventh step 214 the piston rod 128 is moved in the first direction represented by the arrow 304. When the piston rod 128 is moved in the first direction represented by the arrow 304 by actuating the ring 130 to move the piston rod 128 away from the extended position towards the compressed position.

As the piston rod 128 is moved in the first direction represented by the arrow 304, the harpoon structure 132 is inserted into the plunger 142 such that the harpoon structure 132 latches onto the plunger 142. Once the harpoon structure 132 latches onto the plunger 142, the plunger 142 may be moved along with the harpoon structure 132 by moving the ring 130.

As the piston rod 128 is moved in the first direction represented by the arrow 304, the carpule tip 116 pierces fully through the diaphragm 144 of the stopper 140 such that the carpule tip 116 is present within the medication cavity 146 within the carpule 104 such that the carpule tip 116 is in fluid communication with a medication present within the medication cavity 146. For example, when a medical practitioner or user of the injection system 100 moves the piston rod farther away from the extended position to the compressed position, the plunger 142 applies pressure to the medication in the medication cavity 146 causing the medication to enter the carpule tip 116 of the needle 110, pass through the needle 110, and exit through the injection tip 112 of the needle 110. Once the carpule tip 116 is present within the medication cavity 146 of the carpule 104 and the harpoon structure is engaged with the plunger 142, the carpule 104 may be held in place by the harpoon structure 132, may be held in place by one or more internal surfaces of the syringe 102, may be held in place by one or more internal surfaces of the cylindrical body 118, may be held in place by the needle 110, or may be held in place by a combination of these components and structures such that the carpule 104 will not fall out of the syringe 102 when in use.

After the seventh step 214 in which the carpule tip 116 pierces the diaphragm 144 and is present within the medication cavity 146 and the harpoon structure 132 is engaged with the plunger 142 and is latched onto the plunger 142, the injection system 100 is ready for use. In this ready for use state, the cylindrical body 118 fully and completely covers the injection tip 112 of the needle 110 to reduce the likelihood of needlestick injuries when picking up the injection system 100, accidentally dropping the injection system 100, or some other similar or like scenario or event that may result in a needlestick injury.

To inject a patient with the medication in the medication cavity of the carpule 104 after the method of preparing the injection system 100 has been carried out as shown in FIGS. 10 and 11A-11G, the medical practitioner or user may move the cylindrical body 118 of the safety device 108 from the locked and extended position to the retracted position, inserting the injection tip 112 of the needle 110 into a desired location of the patient, and moving the ring 130 to move the piston rod away from the extended position towards the compressed position in the first direction represented by the arrow 304 injecting the patient with the medication. Once the patient has been injected with the medication, the medical practitioner or user may remove the injection tip 112 of the needle 110 from the patient, and then move the cylindrical body 118 of the safety device 108 from the retracted position to the locked and extended position as shown in FIG. 2C to reduce the likelihood of a needlestick injury when the injection system 100 is not in use by the medical practitioner or user.

In the above injection process of the medical practitioner or user utilizing the injection system 100, the detent balls 129a, 129b, 129c, 129d being inserted and removed from the detent openings 127a, 127b may provide an audible “click” sound and/or tactile feedback (e.g., physical click, vibration, etc.) such that the medical practitioner or user is aware of the position of the cylindrical body 118 of the safety device 108 and knows that the cylindrical body 118 will be held in place by one of the detent balls 129a, 129b, 129c, 129d being present within the detent openings 127a, 127b, respectively. This audible sound and/or tactile feedback (e.g., physical click, vibration, etc.) provides the medical practitioner or user confidence in knowing the position of the cylindrical body 118 to avoid the cylindrical body 118 not being fully within the locked and retracted position as shown in FIG. 2A or the locked and extended position as shown in FIG. 2C. In some embodiments, the needle safety sheath, guard, or device 108 may include one or more visual indicators (e.g., colored indicators) that may be visible on an exterior of cylindrical body 118 such that a medical practitioner utilizing the needle safety sheath, guard, or device 108 may quickly determine a position of the needle safety sheath, guard, or device 108. For example, first visual indicators may be adjacent to the detent balls 129a, 129b and second visual indicators may be adjacent to the detent balls 129c, 129d. The first visual indicators may be covered by the tab elements 126a, 126b when the cylindrical body 118 is in the retracted position, and the second visual indicators may be covered by the tab elements 126a, 126b when the cylindrical body is in the extended position. In some embodiments, the first visual indicators may be colored red and the second visual indicators may be colored green. However, the colors of the first and second visual indicators may be adapted or changed to other colors such as blue, yellow, or some other colors as desired.

In the embodiment of the method of preparing the injection system 100 as discussed above with respect to FIGS. 10 and 11A-11G, the safety device 108 may be positioned and mounted on the syringe 102 such that at least one of the opposing pathway slots 120a, 120b overlaps the cavity 138 in which the carpule 104 is present when the syringe 102 is loaded with the carpule 104. This allows a medical practitioner or user to easily view the carpule 104 when loaded in the syringe 102 even when partially covered by the safety device 108 such that the medical practitioner or user may determine or gauge the amount of medication still present within the carpule 104 when the injection system 100 is being utilized to inject a patient. This allows the medical practitioner to easily view the carpule 104 when loaded in the syringe 102 even when partially covered by the safety device 108 such that the medical practitioner may monitor and watch an amount of medication that is being injected into a patient when performing an injection procedure.

In view of the above discussion, in some alternative embodiments of the method in the flowchart 200, the carpule 104 may be inserted into the syringe 102 before the needle assembly 106 may be mounted onto (e.g., threadedly engaged) the syringe 102. In other words, the first, second, third, fourth, fifth, sixth, and seventh steps 202, 204, 206, 208, 210, 212, 214 of the flowchart 200 may be reorganized or adapted to install the safety device 108 on the syringe 102, to install the needle assembly 106 on the syringe 102, and to install the carpule 104 within the syringe 102 in various orders and manners in some alternative embodiments of the method in the flowchart 200 of the present disclosure.

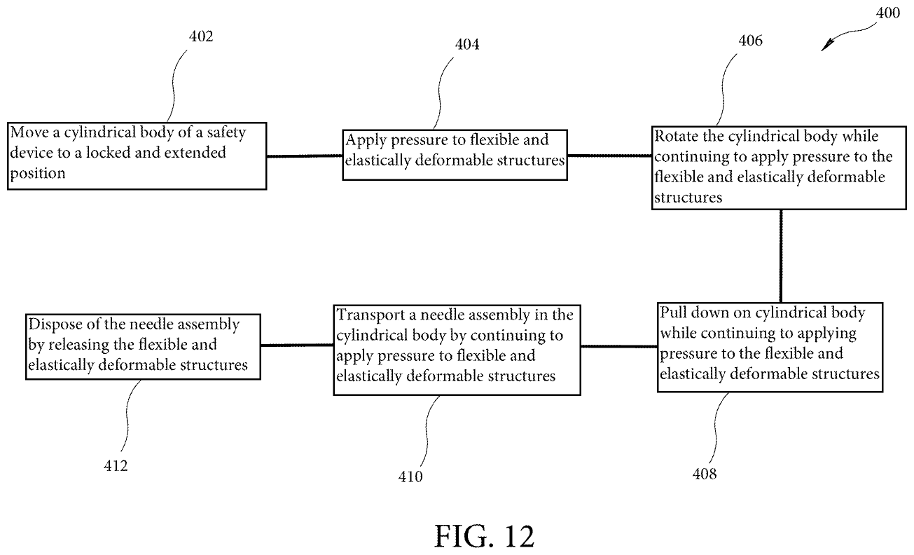

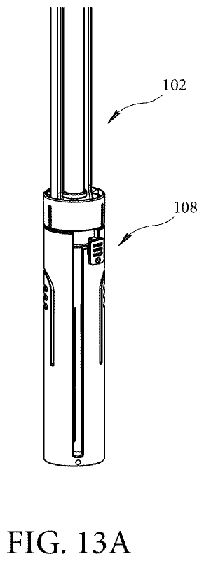

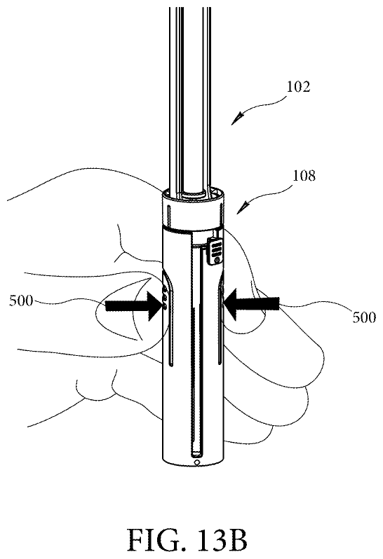

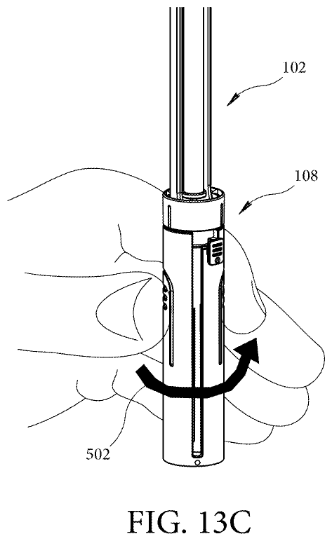

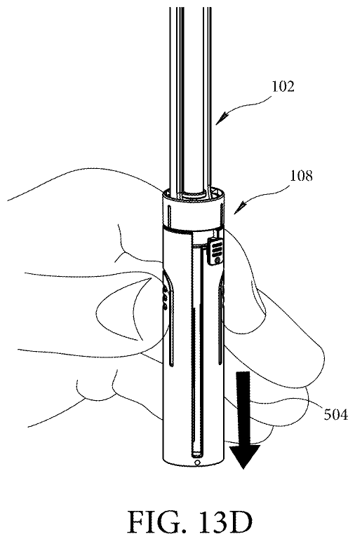

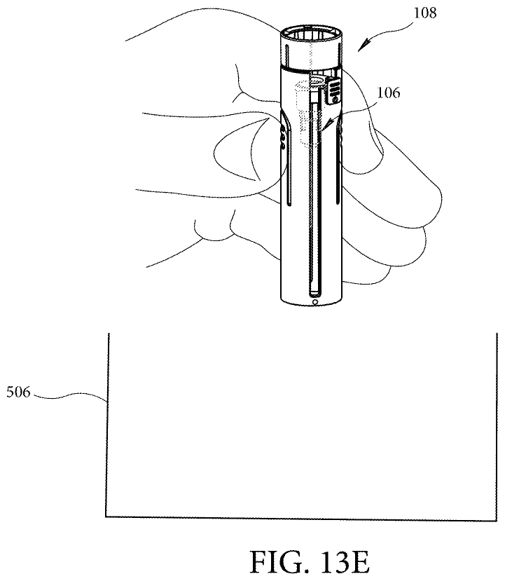

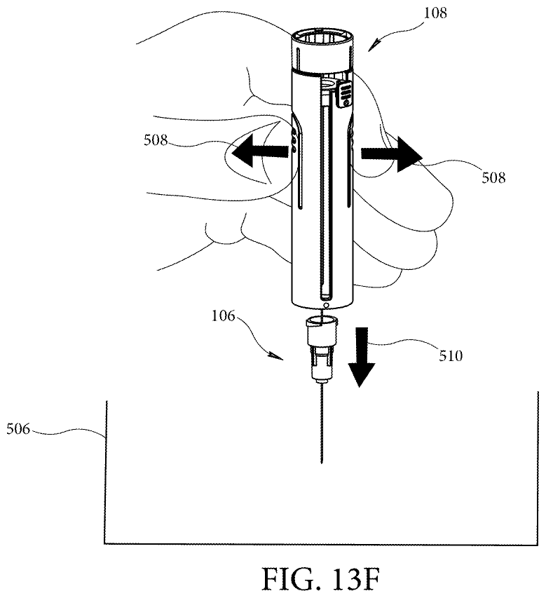

FIG. 12 is directed to a flowchart 400 of an embodiment of a method of disposing the needle assembly 106. The steps of FIG. 12 with respect to the embodiment of the method of disposing of the needle assembly will be discussed with respect to FIGS. 13A-13F. It will be readily appreciated that the steps as shown in the flowchart 400 may be reorganized or completed in a different order than as shown in FIG. 12.

In a first step 402 of the flowchart 400, if the cylindrical body 118 is in the retracted position, the cylindrical body 118 is moved to the locked and extended position in which two of the detent balls 129c, 129d, respectively, are present within the detent openings 127a, 127b, respectively. Once the cylindrical body 118 is in the locked and extended position, the two opposing flexible and elastically deformable grip elements 122a, 122b overlap and are aligned with the needle hub 114 of the needle assembly 106.

After the first step 402 in which the cylindrical body 118 has been moved into the locked and extended position, in a second step 404 a medical practitioner or user applies a pressure to the two opposing flexible and elastically deformable grip elements 122a, 122b. The pressure applied to the two opposing flexible and elastically deformable grip elements 122a, 122b is represented by arrows 500 as shown in FIG. 13B. When the pressure is applied to the two opposing flexible and elastically deformable grip elements 122a, 122b, the two opposing flexible and elastically deformable grip elements 122a, 122b are flexed and deflected inward towards the needle hub 114 of the needle assembly 106. Once enough pressure is applied to the two opposing flexible and elastically deformable grip elements 122a, 122b, the two opposing flexible and elastically deformable grip elements 122a, 122b contact the needle hub 114 of the needle assembly 106 such that the needle hub 114 is pinched between the two opposing flexible and elastically deformable grip elements 122a, 122b, respectively.

After the second step 404 in which the two opposing flexible and elastically deformable grip elements 122a, 122b flexed and deflected inward such that the needle hub 114 is pinched between the two opposing flexible and elastically deformable grip elements 122a, 122b, in a third step 406 the cylindrical body 118 is rotated while continuing to apply pressure to the two opposing flexible and elastically deformable grip elements 122a, 122b such that the needle hub 114 of the needle assembly 106 rotates along with the cylindrical body 118 due to the two opposing flexible and elastically deformable grip elements 122a, 122b pinching onto the needle hub 114 as shown in FIG. 13C. Rotating the needle hub 114 by rotating the cylindrical body 118 while continuing to apply pressure to the flexible and elastically deformable grip elements 122a, 122b threadedly disengages the needle hub 114 of the needle assembly 106 from the threaded structure 134 of the syringe 102. In other words, the needle hub 114 is removed from the threaded structure 134 of the syringe 102. The rotation in the third step 406 is represented by an arrow 502. Alternatively, instead of rotating the cylindrical body 118, the cylindrical body 118 may be held stationary or static while continuing to apply pressure to the flexible and elastically deformable grip elements 122a, 122b as the syringe 102 is rotated such that the needle hub 114 of the needle assembly 106 threadedly disengages with the threaded structure 134 of the syringe 102.

After the third step 406 in which the needle hub 114 is disengaged from the threaded structure 134 by rotating the cylindrical body 118 while continuing to apply pressure to the two opposing flexible and elastically deformable grip elements 122a, 122b to maintain contact with the needle hub 114 such that the needle hub 114 rotates with the cylindrical body 118, in a fourth step 408 the safety device is moved away from the syringe 102 while continuing to apply the pressure to the two opposing flexible and elastically deformable grip elements 122a, 122b such that the needle assembly 106 is held in place within the cylindrical body 118 of the safety device 108 as shown in FIG. 13D. The safety device 108 being moved away from the syringe 102 is represented by an arrow 504 as shown in FIG. 13D.

After the fourth step 408 in which the safety device 108 is moved away from the syringe 102 and the needle assembly 106 is held within the cylindrical body 118 of the safety device 108 by continuing to apply pressure to the two opposing flexible and elastically deformable grip elements 122a, 122b to continue to hold onto the needle hub 114 of the needle assembly 106, in a fifth step 410 the needle assembly 106 is held within the safety device 108 by continuing to apply pressure to the two opposing flexible and elastically deformable grip elements 122a, 122b and is transported to be over a sharps disposal receptacle 506. The sharps disposal receptacle 506 may be a sharps disposal container, sharps disposal bin, or some other disposal structure in which the needle assembly 106 may be disposed as shown in FIG. 13E. In other words, the needle assembly 106 is held within the safety device 108 and is positioned over the sharps disposal receptacle, container, bin, etc. The needle assembly 106 is shown in dotted lines as the needle assembly 106 is present within the cylindrical body 118 of the safety device 108.

After the fifth step 410 in which the needle assembly 106 is held within the safety device 108 and is transported to be over the sharps disposal receptacle 506, in a sixth step 412 the pressure being applied to the flexible and elastically deformable grip elements 122a, 122b is released such that that needle assembly 106 exits the safety device 108 and enters the sharps disposal receptacle 506. In other words, when the pressures on the two opposing flexible and elastically deformable grip elements 122a, 122b is released, the two opposing flexible and elastically deformable grip elements 122a, 122b release the needle hub 114 of the needle assembly 106 (e.g., the needle 110 and the needle hub 114) such that the needle assembly 106 is dropped into the sharps disposal receptacle 506. The releasing of the pressure being applied to the flexible and elastically deformable grip elements 122a, 122b is represented by arrows 508 as shown in FIG. 13F. The disposal or dropping of the needle assembly 106 into the sharps disposal receptacle 506 is represented by the arrow 510 as shown in FIG. 13F.

This disposal of the needle assembly 106 with the safety device 108 allows for the needle assembly 106 to be disposed of in a controlled and safe manner to reduce the likelihood of needlestick injuries while disposing the needle assembly 106 utilizing the method of disposal as shown in the flowchart 400 in FIG. 12. The likelihood of a needlestick injury is reduced utilizing the safety device 108 to dispose of the needle assembly 106 as discussed above with respect to FIGS. 12 and 13A-13F as the injection tip 112 and the carpule tip 116 are fully and completely covered by the cylindrical body 118 of the safety device 108 such that the medical practitioner or user is not exposed to the injection tip 112 or the carpule tip 116 of the needle 110 of the needle assembly 106 when disposing of the needle assembly 106.

In view of the above discussion, the fifth step 410 may be completed at the onset of the method of the flowchart 400 as shown in FIG. 12. For example, when the safety device 108 is positioned in the locked and extended position as shown in FIG. 2C, the syringe 102, the safety device 108, and the needle assembly 106 may be transported to be overlapping or aligned with the sharps disposal container 506 at which point the needle assembly 106 may be removed in the same or similar fashion as discussed above with respect to FIGS. 12 and 13A-13F. In other words, the first, second, third, fourth, fifth, and sixth steps 402, 404, 406, 408, 410, 412 of the flowchart 400 may be reorganized or adapted to dispose of the needle assembly 106 within the sharps container 506 in some alternative embodiments of the method as shown in the flowchart 400 of the present disclosure.

In view of the above discussion with respect to the safety device 108, the safety device 108 reduces or prevents a medical practitioner or user of the injection system 100 from being exposed to the injection tip 112 and the carpule tip 116 of the needle 110 when the safety device 108 is utilized to cover the needle 110 of the needle assembly 106 when the injection system 100 is not in use (e.g., not currently being utilized to inject a patient with a medication within the carpule 104). The safety device 108 also reduces or prevents the medical practitioner or user of the injection system 100 from being exposed to the injection tip 112 and the carpule tip 116 of the needle assembly 106 when disposing of the needle assembly 106. This reduction or prevention of exposure of the medical practitioner or user to the injection tip 112 and the carpule tip 116 reduces or prevents the event of a needlestick injury to the medical practitioner or user.

A needle safety system for dental syringes having a needle cover may be summarized as including a movable member that is exterior to the syringe; one or more features, extending circumferentially outward, integral to the syringe or needle, or on a separate, non-integrated component attached to the syringe or needle; a first position wherein the movable member is configured to expose at least partially the length of the needle attached to the syringe; and a second position, wherein the movable member is configured to surround the tip of the needle attached to the syringe.

The movable member, interfacing with one or more circumferentially extending features, may translate coaxially along the length of the syringe.

Features in the movable member, when in the second position, may prevent substantial movement rotationally, laterally, or axially in the direction of the first position.

The movable member may translate along the length of the syringe between the first and second positions more than one time.

One or more flexible features in the movable member, when in the second position, may deflect radially inward to engage with the hub of the needle attached to the syringe.

The movable member, when engaged with the needle hub, may be rotated for removal from the syringe or the syringe may be rotated for removal from the syringe.

The flexible features in the movable member may disengage the needle hub once removed from the syringe to release the needle from the movable member for proper disposal.

The movable member may be moved out of the second position following needle disposal for sterilization and reuse.

A system may be summarized as including: a syringe; a needle removably on to the syringe, the needle including a tip; a safety guard structure removably mounted to the syringe, the safety guard structure includes a guard sheath, the safety guard structure is separate and distinct from the syringe and the needle, the safety guard structure including: a retracted position in which the tip of the needle is exposed from the safety guard structure configured to provide access to the tip of the needle; and an extended position in which the needle is surrounded by the safety guard structure configured to limit access to the tip of the needle.

The safety guard structure may have a substantially cylindrical shape.

The safety guard structure may be coaxially movable along an exterior of the syringe between the retracted position and the extended position.

The safety guard structure may further include a locked retracted position in which the safety guard structure is locked into place.

The safety guard structure, during operation, may be rotated when in the extended position to rotate the safety guard structure to the locked position.

The safety guard structure may include a locking structure that is configured to lock the safety guard structure in the extended position and in the locked retracted position.

The locking structure may be a ball detent locking structure.

The system may further include a hub structure in which the needle is present, the hub structure is removably coupled to the syringe.

The safety guard structure may further include one or more flexible members that are configured to, during operation, be deflected towards the hub structure in which the needle is present.

The flexible members may be configured to, during operation, mechanically engage with the hub in which the needle is present when the one or more flexible members are deflected towards the hub to remove the hub and the needle from the syringe to dispose of the needle.

The syringe may include a fastening structure at an exterior of the syringe to which the hub is coupled; and the safety guard structure may further include a flange structure that extends around the fastening feature, the flange structure is between the hub and the exterior of the syringe.

The guard sheath may slide along the flange structure between the retracted position and the extended position.

The safety guard structure may include one or more protrusions; and the guard sheath of the safety guard structure may include one or more slots that extend through the guard sheath, each one of the one or more slots receives one of the one or more protrusions, the one or more slots are configured to, during operation, allow the guard sheath to slide along the one or more protrusions between the retracted position and the extended position, respectively.

A method may be summarized as including: removably mounting a safety guard structure separate and distinct from a syringe to an exterior of the syringe; and removably coupling a hub in which a needle is present to a fastening structure at an end of the syringe to overlap a flange structure of the safety guard structure with the hub.

The method may further include removably inserting a carpule into the syringe.

Removably coupling the hub in which the needle is present may further include inserting the needle into a cavity within the carpule.

The method may further include moving a guard sheath of the safety guard structure from a retracted position to an extended position to fully cover a tip of the needle with the guard sheath to prevent access to the tip of the needle.

The method may further include injecting a patient with a medicine within the carpule by inserting the needle into the patient and depressing a plunger of the syringe.

The method may further include removing the needle from the patient; and moving the guard sheath of the safety guard structure from the retracted position to the extended position to fully cover a tip of the needle with the guard sheath to prevent access to the tip of the needle.

The method may further include, when the guard sheath is in the extended position before injecting the patient, moving the guard sheath of the safety guard structure from the extended position to the retracted position.

Removably mounting the safety guard structure separate and distinct from the syringe to the exterior of the syringe further may include removably mounting the safety guard structure in a retracted position to the exterior of the syringe.

A method may be summarized as including: deflecting one or more elastically deformable members along a guard sheath of a safety guard structure removably mounted to a syringe towards a needle hub removably coupled to the syringe; removing the needle hub and the safety guard structure from the syringe while the one or more elastically deformable members are deflected and in mechanical engagement with the needle hub; transporting the needle hub within the safety guard structure and the safety guard structure over an opening in a sharps container while the one or more elastically deformable members are deflected and in mechanical engagement with the needle hub; and dropping the needle hub into the sharps container through the opening of the sharps container by releasing the one or more elastically deformable members to stop the elastically deformable members from being deflected and from being in mechanical engagement with the needle hub.

The method may further include: before removing the needle hub and the safety guard structure from the syringe, injecting a patient with a medication present within a carpule within the syringe by utilizing a needle present within the needle hub, and wherein removing the needle hub from the syringe may include removing the needle along with the needle hub to dispose of the needle hub and the needle by dropping the needle hub and the needle into the sharps container.

The method may further include: transporting the safety guard structure to a sterilization tool; sterilizing the safety guard structure with the sterilization tool; and removably mounting the safety guard structure to the syringe.

A device may be summarized as including: a safety guard structure including: an exterior; a central axis; a sheath portion including a deformable element at the exterior, the deformable element is configured to be deformed towards the central axis; a pathway slot delimited by the sheath portion; an inner member within the sheath portion, the inner member including: an extension that extends into the pathway slot, the extension configured to move along the pathway slot; a tab structure coupled to the extension, the tab structure is accessible at the exterior of the safety device.

The pathway slot may include a first end and a second end opposite to the first end; the sheath portion may further include: a first detent ball at the exterior and adjacent to the first end of the pathway slot; a second detent ball at the exterior and adjacent to the second end of the pathway slot; the inner member may further include a detent opening in the tab structure, the detent opening configured to receive the first detent ball and the second detent ball.

The device may further include: a first locked position in which the first detent ball is in the detent opening; and a second locked position in which the second detent ball is in the detent opening.

The pathway slot may have a J-shape including: a first portion that extends from the first end of the pathway slot; a second portion is transverse to the first portion and extends from the first portion, the second portion is spaced apart from the first end; and a third portion is transverse to the second portion and extends from the second portion to the second end of the pathway slot.

A system may be summarized as including: a syringe; a needle removably on the syringe, the needle including a tip; a safety structure on the syringe, the safety structure includes: a guard sheath; a deformable element at the guard sheath, the deformable element configured to, during operation, be deformed towards the needle; a retracted position in which the tip of the needle is exposed from the guard sheath of the safety structure; and an extended position in which the needle is surrounded by the guard sheath of the safety structure.

The guard sheath may have a substantially cylindrical shape.

The guard sheath may be movable along an exterior of the syringe between the retracted position and the extended position.

The safety structure may include a locking structure that is configured to, during operation, lock the guard sheath in the extended position and in the retracted position.

The locking structure may be a ball detent locking structure.

The locking structure may be a friction fit locking structure.

The locking structure may be a magnetic locking structure.

The system may further comprise a hub structure in which the needle is present, the hub structure may be removably mounted to the syringe.

The deformable element may be configured to, during operation, be deformed to mechanically engage with the hub structure in which the needle is present to remove the hub structure and the needle from the syringe.

The deformable element may be configured to, during operation, be deformed towards the hub structure.

The syringe may include a fastening structure at an exterior of the syringe to which the hub structure may be coupled; and the safety structure further may include a flange structure that extends around the fastening structure of the syringe, the flange structure may be between the hub structure and the exterior of the syringe.

The guard sheath may slide along the flange structure between the retracted position and the extended position.

The flange structure may include an extension; and the guard sheath of the safety structure may include a pathway in the guard sheath, the pathway receives the extension, and the pathway is configured to, during operation, allow the guard sheath to slide along the extension between the retracted position and the extended position.

A method may be summarized as including: deforming one or more elastically deformable structures along a guard sheath of a safety structure removably mounted to a syringe towards a needle assembly removably mounted to the syringe; and removing the needle assembly from the syringe while the one or more elastically deformable structures are deformed and in mechanical engagement with the needle assembly.

The method may further comprise disposing of the needle assembly by releasing the one or more elastically deformable structures.

Removing the needle assembly from the syringe while the one or more elastically deformable structures are deformed and in mechanical engagement with the needle assembly may further include removing the needle assembly from a threaded structure of the syringe.

A device may be summarized as including: a sheath portion including: a deformable element; and a pathway delimited by the sheath portion; an internal member within the sheath portion, the internal member including an extension that mechanically cooperates with the pathway and is configured to, during operation, allow the sheath portion to move along the extension.

The pathway may include a first end and a second end opposite to the first end; the sheath portion may further include: an end; an exterior; a central axis that extends through a center of the sheath portion; a first detent ball at the exterior and adjacent to the first end of the pathway; a second detent ball at the exterior and adjacent to the second end of the pathway; the inner member may further include: a detent opening in the tab structure, the detent opening configured to receive the first detent ball and the second detent ball; the deformable element may be at the exterior of the sheath portion.

The pathway may have a J-shape including: a first end; a second end opposite to the first end; a first portion that extends from the first end of the pathway; a second portion is transverse to the first portion and extends from the first portion, the second portion is spaced apart from the first end; and a third portion is transverse to the second portion and extends from the second portion to the second end of the pathway.

A system may be summarized as including: a syringe; a needle assembly removably on the syringe, the needle assembly including a needle with a tip; a safety structure separate and distinct from the needle assembly on the syringe, the safety structure includes: a guard sheath; a retracted position in which the tip of the needle is exposed from the guard sheath of the safety structure; and an extended position in which the needle is surrounded by the guard sheath of the safety structure.

The guard sheath may have a substantially cylindrical shape.

The guard sheath may be movable along an exterior of the syringe between the retracted position and the extended position.

The safety structure may include a locking structure that is configured to, during operation, lock the guard sheath in the extended position and in the retracted position.

The locking structure may be a ball detent locking structure.

The locking structure may be a friction fit locking structure.

The locking structure may be a magnetic locking structure.

The needle assembly may further comprise a hub structure in which the needle is present, the hub structure may be removably mounted to the syringe.

The system may further comprise an interface element at the guard sheath configured to, during operation, be actuated to mechanically engage with the hub structure.

The interface element may be a deformable element configured to, during operation, be deformed to mechanically engage with the hub structure in which the needle is present to remove the hub structure and the needle from the syringe.

The deformable element may be configured to, during operation, be deformed towards the hub structure.

The syringe may include a fastening structure at an exterior of the syringe to which the hub structure is coupled; and the safety structure may further include a flange structure that extends around the fastening structure of the syringe, the flange structure may be between the hub structure and the exterior of the syringe.

The guard sheath may slide along the flange structure between the retracted position and the extended position.

The flange structure may include an extension; and the guard sheath of the safety structure may include a pathway in the guard sheath, the pathway may receive the extension, and the pathway may be configured to, during operation, allow the guard sheath to slide along the extension between the retracted position and the extended position.

The safety structure may include an interface element at the guard sheath, the interface element may be configured to, during operation, be actuated to remove the needle from the syringe.

A method may be summarized as including: actuating one or more interface structures along a guard sheath of a safety structure removably mounted to a syringe towards a needle assembly removably mounted to the syringe; and removing the needle assembly from the syringe while the one or more interface structures are actuated and in mechanical engagement with the needle assembly.

The method may further comprise disposing of the needle assembly by releasing the one or more elastically deformable structures.

A device may be summarized as including: a sheath including: an interface element; a pathway delimited by the sheath; and an internal member within the sheath portion, the internal member including an extension that mechanically cooperates with the pathway and is configured to, during operation, allow the sheath portion to move along the extension.

The pathway may have a J-shape including: a first end; a second end opposite to the first end; a first portion that extends from the first end of the pathway; a second portion is transverse to the first portion and extends from the first portion, the second portion is spaced apart from the first end; and a third portion is transverse to the second portion and extends from the second portion to the second end of the pathway.

The interface element may be an elastically deformable element.

The various embodiments described above can be combined to provide further embodiments. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A system, comprising:
a syringe;
a needle assembly removably on the syringe, the needle assembly including a needle with a tip;
a safety structure separate and distinct from the needle assembly on the syringe, the safety structure includes:
a guard sheath;
a retracted position in which the tip of the needle is exposed from the guard sheath of the safety structure; and
an extended position in which the needle is surrounded by the guard sheath of the safety structure.

2. The system of claim 1, wherein the guard sheath has a substantially cylindrical shape.

3. The system of claim 1, wherein the guard sheath is movable along an exterior of the syringe between the retracted position and the extended position.

4. The system of claim 1, wherein the safety structure includes a locking structure that is configured to, during operation, lock the guard sheath in the extended position and in the retracted position.

5. The system of claim 4, wherein the locking structure is a ball detent locking structure.

6. The system of claim 4, wherein the locking structure is a friction fit locking structure.

7. The system of claim 4, wherein the locking structure is a magnetic locking structure.

8. The system of claim 1, wherein the needle assembly further comprises a hub structure in which the needle is present, the hub structure is removably mounted to the syringe.

9. The system of claim 8, further comprises an interface element at the guard sheath configured to, during operation, be actuated to mechanically engage with the hub structure.

10. The system of claim 9, wherein the interface element is a deformable element configured to, during operation, be deformed to mechanically engage with the hub structure in which the needle is present to remove the hub structure and the needle from the syringe.

11. The system of claim 9, wherein the deformable element is configured to, during operation, be deformed towards the hub structure.

12. The system of claim 8, wherein:
the syringe includes a fastening structure at an exterior of the syringe to which the hub structure is coupled; and
the safety structure further includes a flange structure that extends around the fastening structure of the syringe, the flange structure is between the hub structure and the exterior of the syringe.

13. The system of claim 12, wherein the guard sheath slides along the flange structure between the retracted position and the extended position.

14. The system of claim 13, wherein:
the flange structure includes an extension; and
the guard sheath of the safety structure includes a pathway in the guard sheath, the pathway receives the extension, and the pathway is configured to, during operation, allow the guard sheath to slide along the extension between the retracted position and the extended position.

15. The system of claim 1, wherein the safety structure includes an interface element at the guard sheath, the interface element is configured to, during operation, be actuated to remove the needle from the syringe.

16. A method, comprising:
actuating one or more interface structures along a guard sheath of a safety structure removably mounted to a syringe towards a needle assembly removably mounted to the syringe; and
removing the needle assembly from the syringe while the one or more interface structures are actuated and in mechanical engagement with the needle assembly.

17. The method of claim 16, further comprising:
disposing of the needle assembly by releasing the one or more elastically deformable structures.

18. A device, comprising:
a sheath including:
an interface element;
a pathway delimited by the sheath; and
an internal member within the sheath portion, the internal member including an extension that mechanically cooperates with the pathway and is configured to, during operation, allow the sheath portion to move along the extension.

19. The device of claim 18, wherein the pathway has a J-shape including:
a first end;
a second end opposite to the first end;
a first portion that extends from the first end of the pathway;
a second portion is transverse to the first portion and extends from the first portion, the second portion is spaced apart from the first end; and
a third portion is transverse to the second portion and extends from the second portion to the second end of the pathway.

20. The device of claim 18, wherein the interface element is an elastically deformable element.
